(19) United States
(12) Patent Application Publication
(10) Pub. No.: US 2025/0340686 A1
(43) Pub. Date: Nov. 6, 2025

(54) ORGANIC ELECTRONIC NOSE USING SEMI-PERMEABLE POLYMER MEMBRANE AND METHOD FOR DETECTING CHEMICAL SPECIES USING THE SAME

(71) Applicants: UNIVERSITY OF SEOUL INDUSTRY COOPERATION FOUNDATION, Seoul (KR); University-Industry Cooperation Group of Kyung Hee University, Yongin-si (KR)

(72) Inventors: Jung Hwa SEO, Seoul (KR); Yu Jung PARK, Seoul (KR); Bright James WALKER, Seoul (KR); Mallory MATIVENGA, Seoul (KR); Jae Young KIM, Seoul (KR); Jae Hyeon LEE, Seoul (KR)

(73) Assignees: UNIVERSITY OF SEOUL INDUSTRY COOPERATION FOUNDATION, Seoul (KR); University-Industry Cooperation Group of Kyung Hee University, Yongin-si (KR)

(21) Appl. No.: 19/196,505

(22) Filed: May 1, 2025

(30) Foreign Application Priority Data
May 2, 2024 (KR) 10-2024-0058358

(51) Int. Cl. C08F 116/06; C08F 8/10; G01N 27/414; G01N 33/00

(52) U.S. Cl. CPC C08F 116/06; C08F 8/10; G01N 27/4141; G01N 33/0047; C08F 2810/20

(57) ABSTRACT

Provided is an organic electronic nose using a semi-permeable polymer membrane and also provided is a method for detecting chemical species using the same, in which the organic electronic nose using a novel polyvinyl alcohol-based semi-permeable polymer membrane can detect and distinguish the chemical species with significantly enhanced selectivity for analytes.

### CROSS-REFERENCE TO RELATED APPLICATION

This application claims priority to Korean Patent Application No. 10-2024-0058358, filed in the Korean Intellectual Property Office on May 2, 2024, the entire contents of which are hereby incorporated by reference.

### BACKGROUND

### Technical Field

The present disclosure relates to an organic electronic nose using a semi-permeable polymer membrane and a method for detecting chemical species using the same, and more specifically, to an organic electronic nose using a semi-permeable polymer membrane that can detect and distinguish chemical species with significantly enhanced selectivity for analytes by using a novel polyvinyl alcohol-based semi-permeable polymer membrane in a chemical sensor, and a method for detecting the chemical species using the same.

### Field

The electronic devices are used in almost every field, including computers, telephones, home appliances, and even clothing. In recent years, with the emergence of the concept of the Internet of Things (IoT), which is an intelligent technology that connects all objects via the Internet to enable mutual information exchange, the world is becoming even more closely connected. For this purpose, accurate detection, communication and control systems have been developed, and the development of sensors has become an indispensable element. A variety of sensors are being developed that can precisely detect light, sound, shape deformation, and so on. However, chemical sensors that detect taste or smell are relatively under-researched and underdeveloped. Electronic nose (E-nose) is a chemical sensor that mimics a biological olfactory sensing process called odor detection, and is formed by combining a number of gas/vapor sensors and a neural network signal processing system (FIG. 1). As various volatile organic compounds (VOCs) or analytes interact with the sensor array, the electronic nose can identify which VOCs are present in the air based on unique response signals from various sensors. However, despite these development efforts, the electronic nose has not yet been commercialized. For commercialization, there are several challenges to overcome, such as high manufacturing costs, low selectivity of sensors, and directivity of circuits.

Since Seyama researchers first used ZnO as a gas sensor in 1960, metal oxide semiconductors (MOSs) such as SnO₂ have been the most widely used in gas detection research and commercially available chemical sensors. The MOS sensors detect vapor phase analytes through changes in electrical signals due to adsorption on MOS surfaces or oxidation-reduction reactions. However, these sensors have very high operating temperatures to enhance sensitivity and selectivity to VOCs such as aromatic compounds. For example, the MOS sensors require a temperature of more than 200° C. to detect alcohols and ketones, and more than 400° C. to detect alkanes. Although this high operating temperature may increase the selectivity of the sensor to some extent, it may be considered a fatal drawback in device applications, and research has been conducted only on a very small number of VOCs. In addition, since MOS materials are mostly insoluble in organic solvents, this is a major factor that complicates the MOS sensor process and makes it difficult to manufacture large-area sensor arrays. For this reason, research on materials and structures that can replace MOS materials has been conducted for decades. Since Shirakawa researchers reported on organic field-effect transistors (OFETs) based on polyacetylene in 1977, many theoretical and experimental studies have been conducted. OFETs have many advantages such as flexibility, low cost manufacturing, and low power driving. In particular, research on OFET is actively being conducted in applications where metal oxide semiconductor field-effect transistors (MOSFETs) cannot be used.

One example of OFET for chemical gas sensor applications is CP FETs, which use conjugated polymers (CPs) as the active material. MOSFET-based gas sensors may detect only VOCs that are adsorbed on the surface of the MOS active layer and cause redox reactions. The MOSFET sensors have very low selectivity for VOCs because various types of volatile organic compounds cause similar electronic effects even when adsorbed on the MOS surface. This implies a lack of diversity of unique VOCs identifiable through MOSFET sensors. As a strategy to solve this problem, the possibility of sensor application of CP FETs is emerging.

In nature, olfactory neurons generate signals through physicochemical interactions between volatile organic compounds (VOCs) and olfactory receptors. These interactions include the volume, shape, polarity of the molecules, aggregation between VOCs and the organic part of olfactory receptors, and so on. For example, a schematic diagram of Eugenol, a volatile substance, interacting with the MhOR5 receptor of insects (Machilis hrabei) is shown in FIG. 2(a). One volatile substance molecule may interact with several olfactory receptors with various affinities, and each receptor may interact with many volatile substances molecules. Various receptors are required to generate an intrinsic neural signal array, and it is known that there are approximately 1000 unique odor receptors in mammals.

The sensing mechanism of the CP FET sensor is fundamentally different from that of the MOSFET sensor, and rather has a closer similarity to the mechanism by which living olfactory neurons operate. In CP FETs, rather than interacting with the surface, VOCs tend to be adsorbed to the detecting layer and then diffuse to the CPs layer. Once the analyte is diffused into an active channel, disturbance and structural changes in π-π stacking that modulates current through semiconductor π-conjugated networks in CP FET channels occur (FIGS. 2(b) and 2(c)). These interactions occur at room temperature and do not depend on irreversible redox reactions between VOCs and CPs. First, analyte diffusion causes changes in the π-π stacking configuration of CPs, which in turn changes the source-drain current (I_{DS}). In CP FETs, several additional phenomena may occur that may affect device behavior, and in particular, as analytes are present in the accumulation layer, a charge trapping phenomenon may occur, which affects the mobility of the device, the threshold voltage, and the current ION/OFF ratio. Van der Waals forces, hydrogen bonding, ionic interactions, hydrophobic-hydrophilic forces, etc. affect the specific interactions that occur between VOCs and CPs, which change charge transport and current through the CP FET device.

These interactions depend not only on the structure of the CP but also on the structure of the VOCs, and may lead to a variety of changes in the electronic properties of OFETs (e.g., mobility (μ), threshold voltage (V_{TH}), current-on/off ratio (I_{ON/OFF}), I_{DS}) compared to MOSFETs. Furthermore, CP FETs have excellent biocompatibility and low operating power and temperature, making them ideal sensors for electronic nose applications. In addition, the unique interactions between CP FETs and VOCs can be said to be similar to the lock-and-key model that forms the basis of odor detection in the animal olfactory system.

An electronic nose may be developed by generating a CP FET array including various sensing materials using CP FETs. By varying the functional groups of CPs based on the backbone of the same polymer, various active materials may be generated that may selectively interact with different molecules and transmit distinct signals when interacting with the different molecules. However, synthesizing CPs with high charge mobility suitable for OFETs is generally a complex and difficult task, and is expensive and has low yields.

Accordingly, as a result of efforts to solve the problems described above and fabricate an organic field-effect transistor (OFET)-based sensor with high selectivity for various chemical species, the inventors of the present disclosure confirmed that, by using a new polyvinyl alcohol-based semi-permeable polymer membrane for a chemical sensor, it is possible to detect and distinguish chemical species of volatile organic compounds (VOCs) including methanol, ethanol, hexane, toluene, tetrahydrofuran, methyl ethyl ketone, and ethyl acetate with significantly enhanced selectivity for analytes, and completed the present disclosure.

### CITATION LIST

### Patent literature

Korea Published Patent Application No. 10-2009-0101906
Korean Registered Patent No. 10-0463677

### SUMMARY

The present invention aims to solve the problems of the related art as described above, and an object of the present invention is to provide a polymer membrane having a high level of excellent selectivity and allowing economic fabrication of a chemical sensor, a chemical sensor using the same, a method for manufacturing the chemical sensor, and a method for detecting various chemical species using the same.

According to an aspect, an acylated polyvinyl alcohol compound represented by Chemical Formula 1 is provided:

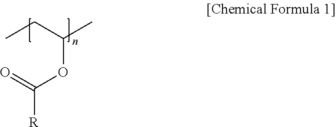

where, R is

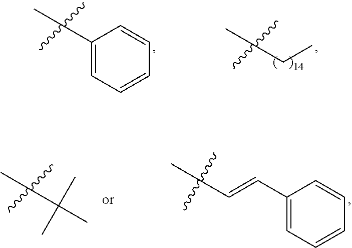

and n is an integer from 50 to 5000.

In addition, according to an aspect, a cross-linked acylated polyvinyl alcohol compound represented by Chemical Formula 2 is provided:

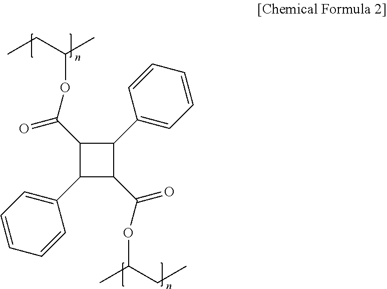

where, n is an integer from 50 to 5000.

In addition, according to an aspect, a method for manufacturing an acylated polyvinyl alcohol compound of Chemical Formula 1 is provided, in which the method includes the steps of: (a) dissolving a polyvinyl alcohol of Chemical Formula 3 in a solvent in a microwave reactor, and (b) adding a base and an acyl chloride of Chemical Formula 4 to the dissolved polyvinyl alcohol and allowing them to react:

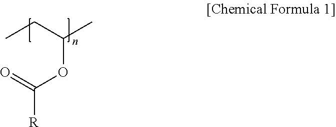

where, R is

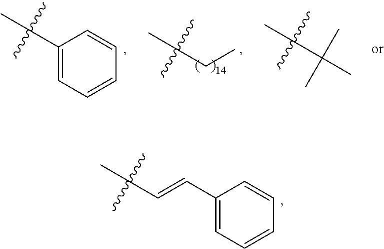

and n is an integer from 50 to 5000,

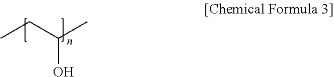

where, n is an integer from 50 to 5000, and

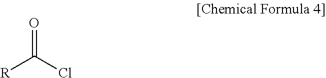

In addition, according to an aspect, a method for manufacturing a cross-linked acylated polyvinyl alcohol compound of Chemical Formula 2 is provided, in which the method includes a step of cross-linking poly (vinyl cinamate) of Chemical Formula 1-4 by irradiating it with UV light:

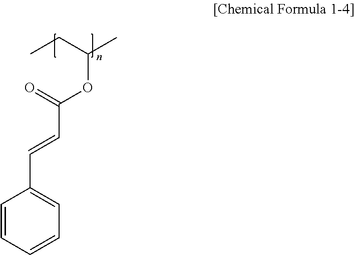

where, n is an integer from 50 to 5000.

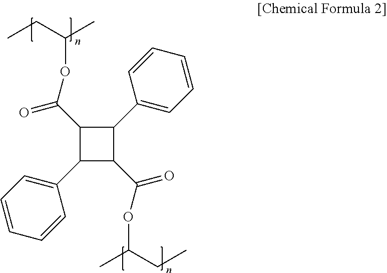

where, n is an integer from 50 to 5000.

In addition, according to an aspect, a chemical sensor is provided, which includes: an organic field-effect transistor (OFET); and a polymer membrane coated with the compound of Chemical Formula 1 or Chemical Formula 2 on the OFET.

In addition, according to an aspect, a method for manufacturing a chemical sensor is provided, which may include a step of coating the OFET with the compound of Chemical Formula 1, or a step of coating the OFET with the compound of Chemical Formula 1-4 and then irradiating it with ultraviolet light.

In addition, according to an aspect, a method for detecting a chemical species using the chemical sensor is provided, which may include a step of bringing the chemical sensor into contact with the chemical species and detecting the chemical species by measuring a drain current over time.

According to the aspects, the chemical sensor using a polyvinyl alcohol-based polymer membrane material can enhance the selectivity for volatile organic compounds (VOCs).

In addition, it is possible to fabricate large-area arrays of organic field-effect transistor (OFET)-based sensors economically.

### BRIEF DESCRIPTION OF THE DRAWINGS

The above and other objects, features and advantages of the present disclosure will become more apparent to those of ordinary skill in the art by describing in detail exemplary aspects thereof with reference to the accompanying drawings, in which:

FIG. 1 is a diagram schematically illustrating how an electronic nose mimics the biological process of olfaction according to an aspect of the present disclosure;

FIG. 2 is a conceptual diagram of an electronic nose according to an aspect of the present disclosure, conceptually representing: (a) the interaction of eugenol with an insect (Machilis hrabei) olfactory neuron (MhOR5) visualized by in-situ TEM [18]; a comparison between (b) the hole transfer of (b) original ordered PBTTT and (c) disordered PBTTT in the presence of toluene; and (d) the use of a semi-permeable membrane layer to enhance the selectivity of the detection layer for certain analytes;

FIG. 3(a) is a diagram showing the synthesis of PVA derivatives: PVBenzoate (1), PVPalmitate (2), PVPivalate (3), and PVCinnamate (4), according to an aspect of the present disclosure; FIG. 3(b) is a diagram showing a reaction equation expressing the cross-linking occurring at the side chains of PVCinnamate after UV irradiation;

FIG. 4 is a schematic diagram showing (a) the structure of a CP FET vapor sensor; and (b) the experimental setup used for vapor sensing test, according to an aspect of the present disclosure;

FIG. 5 is a diagram showing data analysis according to an aspect of the present disclosure, in which FIG. 5(a) shows (a) steps used to extract (ΔI_{DS}/Δt) from raw data; and FIG. 5(b) shows the calculation of exponential decay time constants of various CP FET vapor sensors;

FIG. 6 shows a graph of raw data (I_{DS}) and normalized −ΔI_{DS}/Δt vs. time during hexane detection in (a) a membrane-free CP FET; (b) a PVBenzoate-deposited CP FET; (c) a PVPivalate-deposited CP FET; and (d) a PVPalmitate-deposited CP FET, according to one aspect of the present disclosure;

FIG. 7 is a diagram showing data of a third cycle of −ΔI_{DS}/Δt data of each vapor sensor after exposure to (a) hexane, b) toluene and c) THF analytes according to an aspect of the present disclosure;

FIG. 8 shows ¹H NMR spectra of (a) PVA, (b) PVBenzoate, (c) PVPalmitate, (d) PVPivalate, and (e) PVCinnamate;

FIG. 9 shows the FT-IR spectra of (a) PVA, (b) PVBenzoate, (c) PVPalmitate, (d) PVPivalate, and (e) PVCinnamate, in which the 3300 cm⁻¹ peak (red line) corresponds to the —OH stretching, and the 1703 cm⁻¹ peak (green line) corresponds to the C═O stretching;

FIG. 10 shows the FT-IR spectra (frequency range; 1550 cm⁻¹ to 1850 cm⁻¹) of (a) PVCinnamate, (b) PVCinnamate exposed to UV irradiation (254 nm, 0.27 Ws/cm²) for 15 minutes, and (c) PVCinnamate. UV irradiation (254 nm, 1.08 Ws/cm²) for 60 min. The 1632 cm⁻¹ peak (blue line) corresponds to C═C stretching, the 1703 cm⁻¹ peak (orange line) corresponds to conjugated C═O stretching, and the 1728 cm⁻¹ peak (pink line) corresponds to unconjugated C═O stretching;

FIG. 11 shows the UV-Vis spectra of (a) PVBenzoate, (b) PVPalmitate, (c) PVPivalate, and (d) PVCinnamat;

FIG. 12 is a photograph of a film applicator used to deposit a polymer film having a uniform μm thickness;

FIG. 13 is a diagram showing the relative weight increase (Mt/Mo) compared to analyte exposure time for (a) PVA, (b) PVBenzoate, (c) PVPalmitate, (d) PVPivalate, and (e) PVCinnamate films;

FIG. 14 shows AFM images of (a) PBTTT, (b) PVA, (c) PVBenzoate, (d) PVPalmitate, (e) PVPivalate, and (f) PVCinnamate on a silicon substrate;

FIG. 15 is a diagram showing the source-drain current (I_{DS}) and normalized −ΔI_{DS}/Δt versus time after three cycles of sensing tests for a CP FET vapor sensor without membrane deposition when exposed to (a) methanol, (b) ethanol, (c) hexane, (d) toluene, (e) THF, (f) MEK, and (g) ethyl acetate;

FIG. 16 is a diagram showing the source-drain current (I_{DS}) and normalized −ΔI_{DS}/Δt versus time after three cycles of sensing tests for a CP FET vapor sensor with PVA membrane deposition when exposed to (a) methanol, (b) toluene, (c) THF, and (d) ethyl acetate;

FIG. 17 is a diagram showing the source-drain current (I_{DS}) and normalized −ΔI_{DS}/Δt versus time after three cycles of sensing tests for a CP FET vapor sensor with PVBenzoate membrane deposition when exposed to (a) methanol, (b) ethanol, (c) hexane, (d) toluene, (e) THF, (f) MEK, and (g) ethyl acetate;

FIG. 18 is a diagram showing the source-drain current (I_{DS}) and normalized −ΔI_{DS}/Δt versus time after three cycles of sensing tests for a CP FET vapor sensor with PVPalmitate membrane deposition when exposed to (a) methanol, (b) ethanol, (c) hexane, (d) toluene, (e) THF, (f) MEK, and (g) ethyl acetate;

FIG. 19 is a diagram showing the source-drain current (I_{DS}) and normalized −ΔI_{DS}/Δt versus time after three cycles of sensing tests for a CP FET vapor sensor with PVPivalate membrane deposition when exposed to (a) methanol, (b) ethanol, (c) hexane, (d) toluene, (e) THF, (f) MEK, and (g) ethyl acetate;

FIG. 20 is a diagram showing the source-drain current (I_{DS}) and normalized −ΔI_{DS}/Δt versus time after three cycles of sensing tests for a CP FET vapor sensor with PVCinnamate membrane deposition when exposed to (a) methanol, (b) ethanol, (c) hexane, (d) toluene, (e) THF, (f) MEK, and (g) ethyl acetate;

FIG. 21 is a diagram showing the source-drain current (I_{DS}) and normalized −ΔI_{DS}/Δt versus time after three cycles of sensing tests for a CP FET vapor sensor with cross-linked PVCinnamate membrane deposition when exposed to (a) methanol, (b) ethanol, (c) hexane, (d) toluene, (e) THF, (f) MEK, and (g) ethyl acetate;

FIG. 22 is a diagram showing standardized −ΔI_{DS}/Δt versus time data (third cycle) of all analytes of (a) no membrane, (b) PVBenzoate, (c) PVPalmitate, (d) PVPivalate, (e) PVCinnamate, and (f) cross-linked PVCinnamate deposition CP FET vapor sensor;

FIG. 23 is a diagram summarizing normalized −ΔI_{DS}/Δt versus time data (third cycle) for all types of CP FET vapor sensors when exposed to (a) methanol, (b) ethanol, (c) hexane, (d) toluene, (e) THF, (f) MEK, and (g) ethyl acetate;

FIG. 24 is a diagram showing −ΔI_{DS}/Δt data (third cycle) after sensing methanol, ethanol, hexane, toluene, THF, MEK, and ethyl acetate of (a) CP FET vapor sensor with PVCinnamate deposition and (b) cross-linked CP FET vapor sensor with PVCinnamate deposition;

FIG. 25 is a bar chart summarizing (a) peak dl/dt values and (b) time constant (τ_{decay}) for various analytes detected using various membranes;

FIG. 26 is a diagram showing representative sensor reproducibility data, showing (a) plots of absolute l versus t and normalized dl/dt for the same devices performed during the first test cycle and (b) after 24 h;

FIG. 27 is a diagram showing a transfer curve, an output curve, and extracted parameters, field-effect mobility (μ_{FE}), subthreshold swing (SS), and subthreshold voltage (VTH) of a membrane-free CP FET, (a) before the vapor sensing test and (b) after the vapor sensing test seven times;

FIG. 28 is a diagram showing a transfer curve, an output curve, and extracted parameters, field-effect mobility (μFE), subthreshold swing (SS), and subthreshold voltage (VTH) of a CP FET with a PVBenzoate membrane, (a) before the vapor sensing test and (b) after the vapor sensing test seven times;

FIG. 29 is a diagram showing a transfer curve, an output curve and extracted parameters, field-effect mobility (μFE), subthreshold swing (SS), and subthreshold voltage (VTH) of a CP FET with a PVPalmitate membrane, (a) before the vapor sensing test and (b) after the vapor sensing test seven times;

FIG. 30 is a diagram showing a transfer curve, an output curve, and extracted parameters, field-effect mobility (μFE), subthreshold swing (SS), and subthreshold voltage (VTH) of a CP FET with a PVCinnamate membrane, (a) before the vapor sensing test and (b) after the vapor sensing test seven times;

FIG. 31 is a diagram showing a transfer curve, an output curve, and extracted parameters, field-effect mobility (μFE), subthreshold swing (SS), and subthreshold voltage (VTH) of a CP FET with a PVCinnamate membrane, (a) before the vapor sensing test and (b) after the vapor sensing test seven times;

FIG. 32 is a diagram showing a transfer curve, an output curve, and extracted parameters, field-effect mobility (μFE), subthreshold swing (SS), and subthreshold voltage (VTH) of a CP FET with a cross-linked PVCinnamate membrane, (a) before the vapor sensing test and (b) after the vapor sensing test seven times;

### DETAILED DESCRIPTION

Unless otherwise defined, all technical and scientific terms used herein have the same meanings as those generally understood by those skilled in the art to which the present disclosure pertains. In general, the nomenclature used herein is well known and commonly used in the art.

It is confirmed herein that, when used in chemical sensors, a novel polyvinyl alcohol-based semi-permeable polymer membrane can detect and distinguish chemical species of volatile organic compounds (VOCs), including methanol, ethanol, hexane, toluene, tetrahydrofuran, methyl ethyl ketone, and ethyl acetate with significantly enhanced selectivity for analytes.

In one aspect, electronic noses (E-noses) refer to electronic devices or systems that mimic olfactory organs by quantitatively measuring volatile organic compounds (VOCs) to simulate the sense of smell. The electronic nose may be applied to real-time monitoring of VOCs in various fields such as food, healthcare, and forensics. Related organic field-effect transistor (OFET)-based sensors have the advantage of enabling the economic fabrication of large-area arrays, but research on the selectivity of the sensors, which is required to distinguish various VOCs, has progressed slowly.

Therefore, in one aspect of the present disclosure, a semi-permeable polymer membrane is used in an OFET sensor to enhance selectivity. An acylated polyvinyl alcohol (PVA) derivative was newly synthesized as a polymer membrane material and applied to an OFET vapor sensor to investigate sensing characteristics with and without the membrane polymer layer. By monitoring the drain current over time, the changes in the response rate of the PVA derivative polymer membrane layer to various analytes were observed. All VOCs, including methanol, ethanol, hexane, toluene, tetrahydrofuran, methyl ethyl ketone, and ethyl acetate, can be detected and distinguished with much greater selectivity than by the sensors without the polymer membrane layer. This approach can fabricate various organic vapor sensors more economically simply by changing the membrane layer and provide higher level of selectivity for OFET vapor sensors.

In one aspect, sensing process is divided into two stages: 1) adsorption of VOCs; and 2) detection of analytes. By stacking a thin, semi-permeable membrane polymer over the sensing CP layer, the selectivity of the sensor array can be increased without performing complex CP synthesis. Recently, studies using membrane layers to enhance the selectivity of metal oxide gas sensors have been reported; however, research cases applying this approach to CP FETs are very rarely found [25]. The role of the membrane is to modulate the electrical response signal for different analytes by allowing the specific type of analyte to pass while blocking other types of VOCs to be sensed [25]. In addition, since the diffusion of analytes through the membrane is slower than that in the air [26 to 28], the saturation of analytes in the detecting layer is delayed, thereby delaying the response time and peak signal, enabling quantitative analysis. A variety of polymer membrane designs with unique temporal responses are possible depending on their chemical structure.

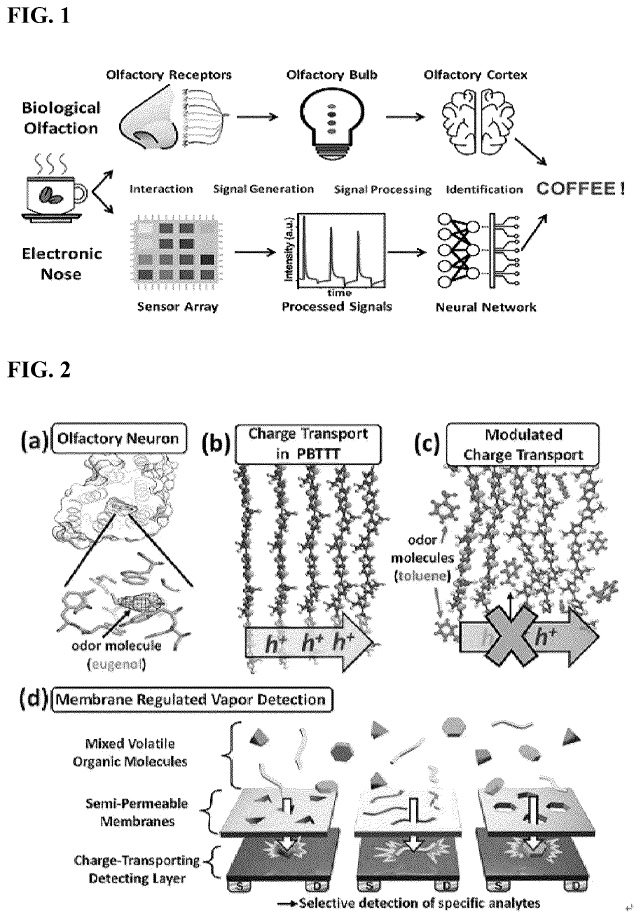

Although there is a potential utility of the dual structure of membrane layers/detecting layers, studies on this topic have been rarely addressed in the field of CP FET vapor sensor research. Therefore, an aspect of the disclosure relates to a study on enhancing the selectivity of a CP FET vapor sensor by using the membrane. As a strategy for designing a membrane, an aspect of the present disclosure involves binding various functional groups to a basic polymer backbone, thereby imparting new functional groups that enable unique interactions with analytes. The aim is to enhance selectivity by selecting a polymer backbone material with low permeability and utilizing reactions with analytes that have structures capable of interacting effectively with the bonded fuctional groups. This concept is schematically illustrated in FIG. 2(d).

Polyvinyl alcohol (PVA) is selected as the polymer backbone material and esterified with various acyl chloride to impart functionality. PVA itself is known as an excellent shield for organic molecules, and hydrogen bonds between abundant hydroxyl groups (—OH) in PVA provide excellent chemical resistance to nonpolar VOCs and strong mechanical properties [29,30]. Moreover, hydroxyl groups react readily with either acid chloride or carboxylic anhydride to form esters. Therefore, functional polymers can be easily synthesized through various esterification reactions using various commercial anhydrides and acyl chloride [31 to 33]. Although certain geometric holes are depicted in the schematic view of FIG. 2(d), the membrane investigated in this study does not include specific geometric holes nor modulate vapor transport via penetration, and instead, acts through diffusion and modulates vapor transport based on chemical interactions between analytes and the functional groups within the membrane.

The chemical properties of the synthesized PVA derivatives depend on the functionalization agent and the degree of functionalization, which allows coordination of the interaction between analytes and membranes. Seven analytes (VOCs) of methanol, ethanol, hexane, toluene, tetrahydrofuran (THF), methyl ethyl ketone (MEK), and ethyl acetate (EA) were used to demonstrate the effect of the membrane on the CP FET. In one aspect, five PVA membranes and one conjugated organic semiconductor (PBTTT) are used as CP FET materials, but it is to be noted that the present method allows for the combination of a wider variety of polymer membranes and polymer organic semiconductors, which enables the fabrication of a large-scale unique vapor sensor array capable of identifying a wider range of VOCs. When combined with appropriate signal processing and machine learning software that correlates the electrical response patterns of the sensor array with specific analytes or odors, it is expected that a functional and practical electronic nose can ultimately be implemented.

Therefore, in one aspect of the disclosure, an acylated polyvinyl alcohol compound represented by Chemical Formula 1 is provided:

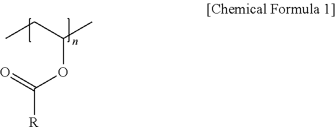

where, R is

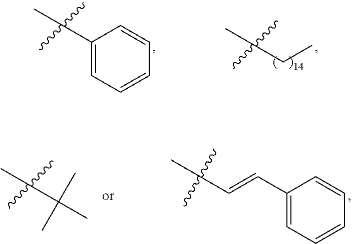

and n is an integer from 50 to 5000.
where, when R is

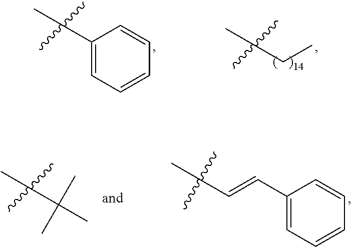

the compound names are poly(vinyl benzoate) (PVBenzoate), poly(vinyl palmitate) (PVPalmitate), poly(vinyl pivalate) (PVPivalate), and poly(vinyl cinamate) (PVCinnamate).

In addition, in one aspect, n is a variable representing the degree of polymerization in the polymer, and n is an integer of 50 to 5000, preferably an integer of 200 to 5000, and more preferably an integer of 2000 to 2900.

In one aspect, a cross-linked acylated polyvinyl alcohol compound represented by Chemical Formula 2 is provided:

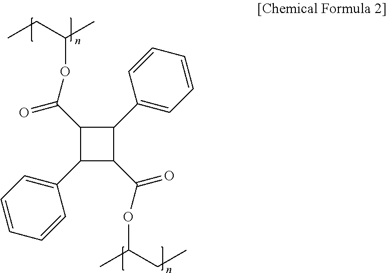

where, n is an integer from 50 to 5000.

Further, in another aspect, a method for manufacturing the acylated polyvinyl alcohol compound of Chemical Formula 1 is provided, in which the method includes the steps of: (a) dissolving a polyvinyl alcohol of Chemical Formula 3 in a solvent in a microwave reactor, and (b) adding a base and an acyl chloride of Chemical Formula 4 to the dissolved polyvinyl alcohol and allowing them to react:

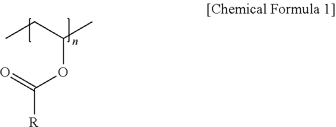

where, R is

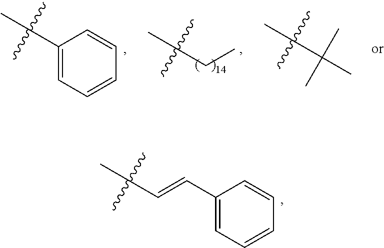

and n is an integer from 50 to 5000.

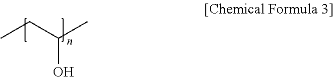

where, n is an integer from 50 to 5000.

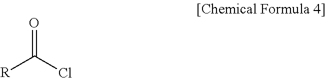

In one aspect, the solvent of step (a) may be at least one selected from the group consisting of 1-methylimidazole, N-methylpyrrolidone, 1-methylimidazole, N-methylpyrrolidone, dimethyl formamide (DMF), and dimethyl sulfoxide (DMSO).

In one aspect, a reaction time in step (b) may be 5 minutes to 24 hours, in which 30 minutes to 3 hours is the most ideal reaction time, and if the reaction time is short, there is a possibility that some of the chemical reactions will not occur, and if the reaction time is too long, polymer aggregation occurs due to solubility change due to evaporation of the solvent. When polymers aggregate, it may cause difficulties in forming the membrane.

In still another aspect of the present disclosure, a method for manufacturing a cross- linked acylated polyvinyl alcohol compound of Chemical Formula 2 is provided, in which the method includes a step of cross-linking the poly (vinyl cinamate) of Chemical Formula 1-4 by irradiating it with UV light:

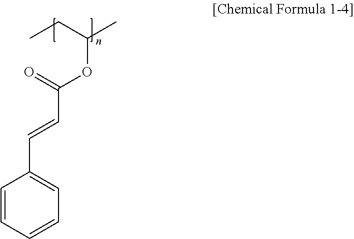

where, n is an integer from 50 to 5000.

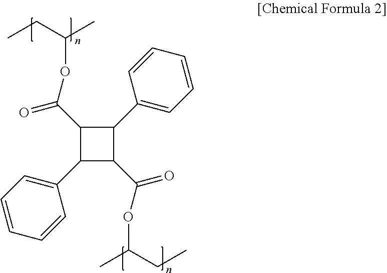

where, n is an integer from 50 to 5000.

In general, it has been reported in this field that cross-linking occurs when UV light at 254 nm or in the range of 310-380 nm is irradiated. Since the commercially available UV light irradiation devices emit light at 254 nm, the process was carried out at this wavelength, but cross-linking may be formed within the range of 120 nm to 380 nm.

In still another aspect, a chemical sensor is provided, which includes: an organic field-effect transistor (OFET); and a polymer membrane coated with the compound of Chemical Formula 1 or Chemical Formula 2 on the OFET.

In one aspect, the polymer membrane may have a thickness of 50 nm to 1 μm. If the polymer membrane is thicker than this thickness, the response speed of the sensor will decrease, and if the membrane is thinner, the membrane effect will be diminished, resulting in reduced sensor sensitivity.

In still another aspect, a method for manufacturing a chemical sensor is provided, in which the method includes a step of coating OFET with the compound of Chemical Formula 1. The coating may include spin coating, drop casting, dip coating, blading coating, spray coating, or 3D printing, and preferably, spin coating is performed.

In still another aspect, a method for manufacturing a chemical sensor is provided, in which the method includes a step of coating an OFET with the compound of Chemical Formula 1-4 and then irradiating it with ultraviolet light.

In one aspect, after the step of coating, the method may further include a step of performing heat treatment at a temperature of 80 to 150° C. for 15 to 30 minutes.

In still another aspect, a method for detecting a chemical species using a chemical sensor is provided, in which the method includes a step of bringing the chemical sensor into contact with the chemical species and detecting the chemical species by measuring a drain current over time.

In one aspect, the chemical species includes volatile organic compounds (VOCs) such as methanol, ethanol, hexane, toluene, tetrahydrofuran, methyl ethyl ketone, and ethyl acetate.

Hereinafter, the present disclosure will be described in more detail with reference to examples. These examples are intended only to illustrate the present disclosure, and it will be apparent to those skilled in the art that the scope of the present disclosure is not interpreted as being limited by these examples.

### EXAMPLES

### Example 1: Synthesis of Acylated PVA Derivative

### Experimental Materials

Polyvinyl alcohol (PVA) (99+%, hydrolyzed, 89,000 to 124,000 MW) was purchased from Aldirch. Benzoyl chloride, palmitoyl chloride, chlorobenzene, and 1-methyl imidazole were purchased from Tokyo Chemical Industry Co., Ltd. Cinnamon chloride, pyridine, butyl acetate, and propylene glycol monomethyl ether acetate (PGMEA) were purchased from Acros Organics. Pivaloyl chloride, N-methyl-2-pyrrolidone (NMP), and other chemicals for vapor detection were purchased from Daejeong Chemical. Poly[2,5-bis(3-tetradecylthiophene-2-yl)thieno[3,2-b]thiophene] (Poly[2,5-bis(3-tetradecylthiophene-2-yl)thieno[3,2-b]thiophene], PBTTT-C14) was purchased from 1-Material Organic Nano Electronic. Air-sensitive reagents were stored in glove boxes filled with nitrogen. The organic solvent was completely dried with 3A molecules.

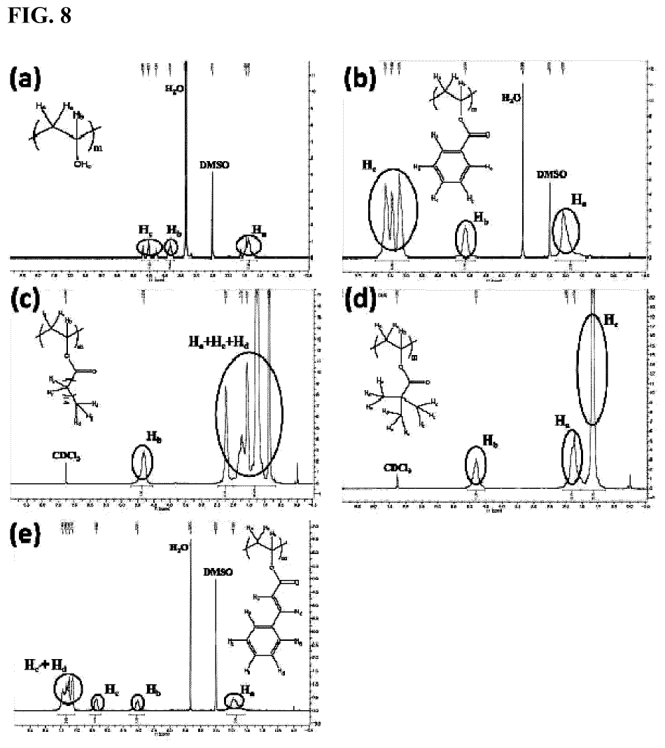

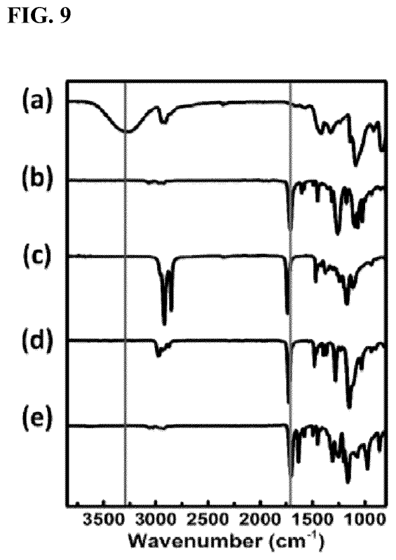

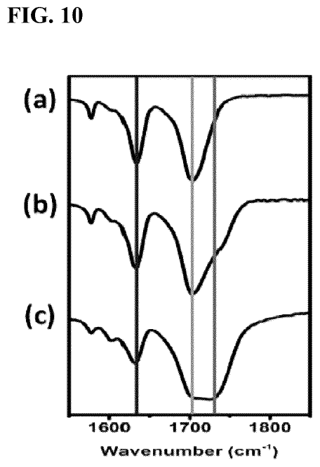

Each PVA solution was prepared using a microwave reactor to significantly reduce the time required to dissolve PVA in each solvent. It took 1 h to completely dissolve PVA in NMP, and 30 min at 105° C. in 1-methyl imidazole. At each reaction temperature, a base was added immediately before the addition of acyl chloride. After each reaction was complete, PVBenzoate and PVCinnamate solutions were precipitated from methanol and then dissolved in chlorineform and precipitated again from methanol. after the reaction was complete, the PVPivalate reaction mixture was precipitated from water and then redissolved in THF and precipitated again from water. The PVPalmitate solution was precipitated from methanol and then redissolved in THF and precipitated again from methanol. Each polymer was purified by sequentially redissolving and precipitating three times from an appropriate solvent/antisolvent system as described above. Details of the respective reaction conditions are shown in FIGS. 8 to 11, and details of the reaction conditions are summarized in Table 1.

TABLE 1
Synthesis Conditions, Degree of Functionalization,
and Yield of Acylated PVA Derivatives

| | PVA:RCOCl | | | Rxn | Rxn | Degree of | |
|---|---|---|---|---|---|---|---|
| # | Ratio | Solvent | Base | Temp | Time | Functionalization* | Yield |
| 1 | Benzoyl | 1-methyl | — | 105° C. | 30 min | 100% | 85.6% |
| | Chloride | imidazole | | | | | |
| | Eq: 1.5 | 2-5 wt % | | | | | |
| 2 | Pivaloyl | NMP | 1-methyl | 105° C. | 30 min | 100% | 89.8% |
| | Chloride | 3 wt % | imidazole | | | | |
| | Eq: 1.5 | | Eq: 2 | | | | |
| 3 | Palmitoyl | NMP | Pyridine | 105° C. | 30 min | 100% | 78.5% |
| | Chloride | 3 wt % | Eq: 2.3 | | | | |
| | Eq: 1.8 | | | | | | |
| 4 | Cinnamoyl | NMP | Pyridine | 95° C. | 60 min | 100% | 70.1% |
| | Chloride | 2 wt % | Eq: 2.3 | | | | |
| | Eq: 1.8 | | | | | | |

*Based on the complete disappearance of the OH signal in FTIR, 1HNMR, and UV-Vis.

### Swelling Test of Acylated PVA Derivative

An experiment was conducted to calculate the diffusion coefficient of the volume change experiment of acylated PVA in various analytes (VOCs) (FIG. 12). A uniform thin film of PVA derivative was prepared using a commercial film applicator. For this purpose, an aluminum substrate and a flat glass plate were used. The thickness of the thin film was measured using a digital micrometer (Mitutoyo), and the mass was measured using a digital scale. For the volume change experiment, the thin PVA polymer film samples were fixed between aluminum meshes, placed in separate vials, and closed with a lid. The total mass of each vial was also measured before the volume change experiment. Lidless vials were placed in large containers filled with atmospheres saturated with vapors of different analytes for each sample. After 15 minutes of exposure, the vials were sealed with lids and removed from the large containers to measure their total mass. The amount of vapor absorbed by the thin PVA polymer film sample over the 15-minute period can be quantified for each analyte by subtracting the initial mass from the final mass. After the initial measurement, the increased mass was re-measured at regular time intervals until the total time reached 600 minutes, at which time it was observed that most of the thin film samples changed in volume and were completely dissolved in solvent vapor.

### Example 2: Device Fabrication

### FET Fabrication

The FET was fabricated on a glass substrate with an inverted coplanar structure. In the fabrication process, a 40 nm-thick Mo layer was deposited by sputtering at 280° C. and patterned to form a gate electrode. Then, a 40 nm-thick AlOx layer was spin-coated on the gate to form a thin film layer. AlOx solution (0.2 M) was prepared by mixing AlCl₃, acetonitrile (35%), and ethylene glycol (65%). Each of the two layers was spin-coated with AlOx solution at 2000 rpm for 30 s, resulting in a total thickness of 40 nm. The AlOx layer was cured at 250° C. for 5 minutes to evaporate the solvent, and then subjected to UV ozone treatment for 5 minutes. For thin film densification, the final heat treatment step was performed at 350° C. for 2 hours under vacuum. For the source and drain electrodes, a 60 nm-thick IZO layer was sputter-deposited at 280° C. and patterned by a wet etching process. A 1.3 μm-thick bipolar PR layer was spin-coated over the source and drain electrodes and patterned to form a “bank”. After the bank formation, the device was ultrasonically cleaned in isopropyl alcohol for 10 minutes, followed by rinsing with deionized water. Finally, an organic semiconductor layer was formed by spin coating.

### Preparation of PBTTT and PVA Derivative Solutions and Thin Film Fabrication

PBTTT was dissolved in anhydrous benzene chloride (3 mg/ml) and deposited on FET substrates by spin coating at 2000 rpm for 60 s at 120° C. in a vacuum-filled state in a vacuum chamber. Each membrane layer has a specific geometry and operates via diffusion rather than regulating gas transport, and regulates vapor transport based on chemical interactions with compounds within the membrane.

Each membrane was dissolved in an appropriate organic solvent and formed into a thin film by spin coating. PVA was dissolved in distilled water (48 mg/ml) and spin-coated in air on OFET at 5000 rpm for 60 s at room temperature. Membranes of PVBenzoate and PVPivalate were spin-coated at 2000 rpm for 60 s at room temperature using polymer solutions dissolved in anhydrous acetonitrile (48 mg/ml) in a vacuum chamber. PVPalmitate membranes were prepared using distilled water (96 mg/ml) on a substrate in the same manner. Thin PVCinnamate membranes were made with anhydrous PGMEA (72 mg/ml) before spin coating, and the solution was heated to 100° C. The spin coating conditions for PVCinnamate were the same as those for the other PVA derivatives. All deposited thin films, including PVCinnamate, were heat-treated at 120° C. for 20 min after spin coating. The PVCinnamate film was heat-treated at 100° C. for 20 min. Details of the spin coating conditions and film thickness are shown in Table 2. The surface and roughness of each film were examined by atomic force microscopy (AFM). The PVCinnamate film cross-linked on the CP FET sensor was fabricated by irradiating it with ultraviolet light at 80° C. for 1 hour. The hand-type UV lamp has an intensity of 300 μW/cm² and a total UV exposure dose of 1.08 Ws/cm² at a distance of 15 cm from the thin film for 60 minutes.

TABLE 2
Polymer Spin Coating Conditions

| | | Concentration | Solution | Spin | Spin | Annealing | Annealing | Thickness |
|---|---|---|---|---|---|---|---|---|
| Polymer | Solvent | (mg/ml) | Temperature | Rate | Time | Temperature | Time | (nm) |
| PBTTT | Chloro- | 3 | 120° C. | 2000 rpm | 60 s | 120° C. | 20 min | 40.5 |
| | benzene | | | | | | | |
| PVA | Distilled | 48 | Room Temp. | 5000 rpm | 60 s | 120° C. | 20 min | 194.5 |
| | Water | | | | | | | |
| PVBenzoate | Butyl | 48 | Room Temp. | 2000 rpm | 60 s | 120° C. | 20 min | 158.5 |
| | Acetate | | | | | | | |
| PVPivalate | Butyl | 48 | Room Temp. | 2000 rpm | 60 s | 120° C. | 20 min | 388.5 |
| | Acetate | | | | | | | |
| PVPalmitate | Butyl | 96 | Room Temp. | 2000 rpm | 60 s | 120° C. | 20 min | 702.5 |
| | Acetate | | | | | | | |
| PVCinnamate | PGMEA | 72 | 100° C. | 2000 rpm | 60 s | 100° C. | 20 min | 316.5 |

### Example 3: Synthesis and Characterization of Acylated PVA

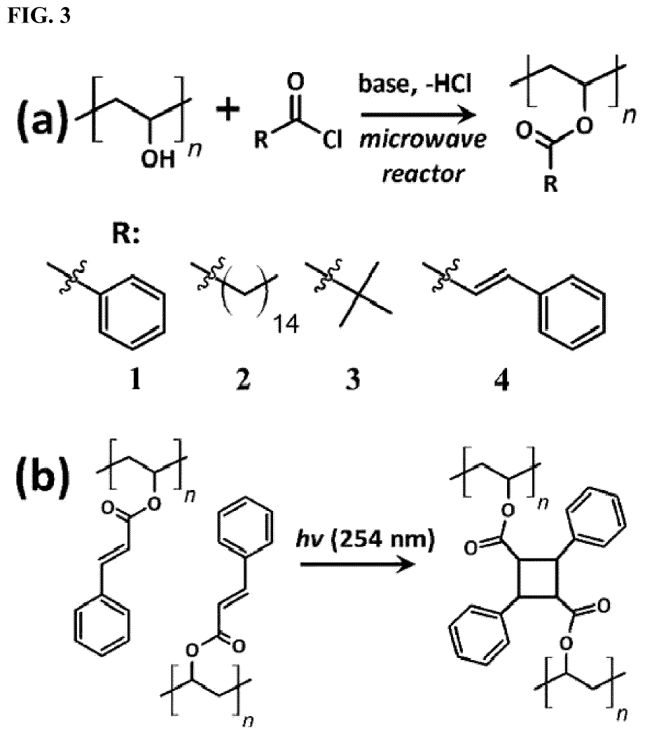

The synthesis of poly(vinyl benzoate) (PVBenzoate) (1), poly(vinyl palmitate) (PVPalmitate) (2), poly(vinyl pivalate) (PVPivalate) (3), and poly(vinyl cinnamate) (PVCinnamate) (4) is shown in FIG. 3(a). Because PVA has a strong hydrogen bonding between the hydroxyl groups, it is insoluble in most organic solvents and is almost 100% etherified. To reduce the complexity and time required for PVA synthesis, the Eastman research team reported the use of 1-butyl-3-methylimidazolium chloride as the solvent for the esterification reaction and described the reaction between various acid chlorides and PVA. As a result, the reaction time was significantly reduced to about 10-15 minutes, and an etherification conversion of over 95% of the hydroxyl groups was demonstrated. However, it still took 18 to 48 hours to dissolve PVA completely, and the synthesis of ionic fluids complicating this reaction was required [32].

In one aspect of the present disclosure, the acceleration reaction is enhanced rapidly and economically using a microwave reactor. It took only 30 to 60 min to completely dissolve PVA in 1-methylimidazole and N-methylpyrrolidone by microwave irradiation. In addition, the microwave-accelerated reaction uniformly heats the reaction solution, directly induces the acylation reaction, and accelerates collisions, enabling consistent achievement of 100% acylation of PVA. The cinamate substrate may be cross-linked when exposed to UV light [34-36] (FIG. 3(b)). Therefore, another polymer with new characteristics may be produced by spin-coating the PVCinnamate film and irradiating it with 254 nm UV light as described in the experimental part. This cross-linked thin film, like other PVA derivatives, may be used as a membrane for CP FETs. Detailed reaction conditions are presented in the experimental method. The properties of 100% functionalized PVA derivatives, cross-linked PVCinnamate, etc. were further investigated through NMR, FT-IR and UV-Vis spectrometers (FIGS. 8 to 11).

### Comparison of 7 Different Analytes and Expansion (Swelling) Experiments of Acylated PVA

First, various organic solvents (VOCs) were used as analytes to investigate the selectivity of the vapor sensor. Methanol and ethanol are relatively polar VOCs with OH groups, while the other solvents have similar Hansen solubility parameters and possess chemical structures similar to ethanol with additional CH₂ groups. These analytes were selected to assess how effectively CP FET sensors with the membranes can distinguish molecules with similar structures.

Hexane is a nonpolar, linear, aliphatic molecule with poor solubility to PBTTT [37]. If the CP FET responds to hexane without a membrane, this indicates that the PBTTT sensor can detect nonpolar, aliphatic VOCs, which are characteristic of some odor molecules. In addition, the hexane sensing tests are expected to demonstrate that comparing the results of PVPalmitate-CP FET and other membrane-CP FET sensors show how the compound structure similarity between the membrane and the analyte can enhance the selectivity of the vapor sensor.

Toluene was selected as an analyte to determine whether there was a unique effect due to the conjugated aspects of PBTTT or PVCinnamate and π-π interactions. This may cause different signal changes compared to other analytes.

THF is the only analyte that is a good solvent for dissolving PVA derivatives synthesized with PBTTT. Therefore, THF may damage CP FET sensors or membranes, and stability tests were conducted by evaluating the characteristics of sensors after THE detection.

MEK and EA have similar compound structures except that there is one more oxygen atom. This corresponds to the ketone and ester functional groups often found in odor molecules. Comparing the results of these analytes could demonstrate the ability of sensors to distinguish analytes that have similar functional groups.

The diffusion coefficient of a specific vapor molecule quantifies the rate at which the molecule passes through and permeates the polymer. Therefore, the diffusion coefficient provides information about how a specific membrane interacts with a specific analyte. A high diffusion coefficient indicates that the analyte should rapidly pass through the membrane and interact with the detecting layer, while a diffusion coefficient of zero indicates that the corresponding type of analyte should be blocked.

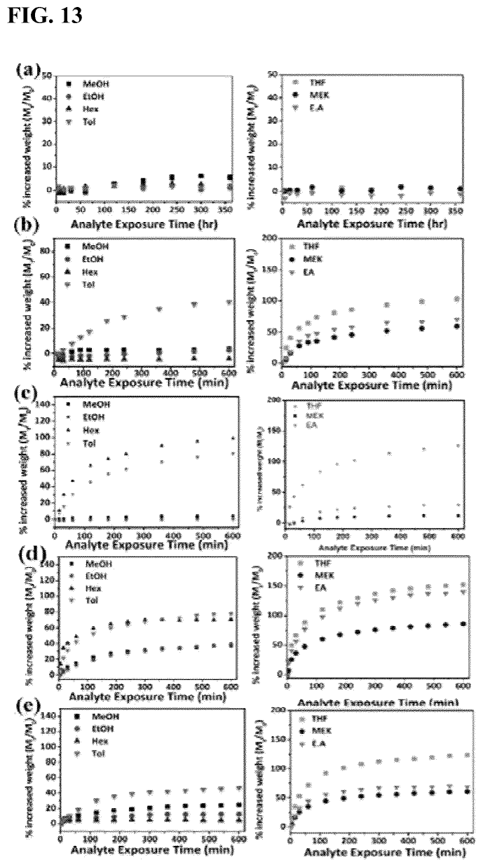

To measure the diffusion coefficient, a simple gravimetric analysis can be used, in which a uniform polymer film is exposed to analyte vapor and the degree or rate of swelling of the film over time is recorded. The diffusion coefficient was extracted from the swelling data using the Half Time method (Equation 1) [26-28]. Details of the experiment are included in the experimental method section, and the results of the swelling experiment are shown in FIG. 13 and Tables 3 to 8. However, in practice, no meaningful correlation was observed between the diffusion coefficients measured through this technique and the vapor detection results observed in the examples of the present disclosure.

$$D ≅ ( 0.04919 ) \times ( \frac{{h}^{2}}{{t}_{1 / 2}} ) \qquad [ Mathematical Expression 1 ]$$

where, D is a diffusion coefficient, h is a thickness of the polymer sheet, and t½ is a time (t) when Mt/M600 is ½.

TABLE 3
Relative Weight Increase of PVA (Mt/Mo)
After Exposure to Each Analyte Over Time

| Unit: % | | Methanol | Ethanol | Hexane | Toluene | THF | MEK | Ethyl Acetate |
|---|---|---|---|---|---|---|---|---|
| 0 | mins | 0.0 | 0.0 | 0.0 | 0.0 | 0.0 | 0.0 | 0.0 |
| 5 | mins | −1.0 | 0.6 | 0.1 | 1.7 | −0.1 | 0.3 | −2.9 |
| 15 | mins | −1.0 | 0.3 | 0.7 | 0.6 | 0.1 | 0.6 | −0.8 |
| 30 | mins | −0.1 | 0.9 | 0.4 | 1.1 | 0.2 | 0.6 | −1.1 |
| 60 | mins | −0.7 | 0.6 | 1.8 | 1.1 | 0.1 | 1.9 | −0.8 |
| 120 | mins | 3.1 | 2.6 | 1.8 | 2.3 | 1.5 | 0.3 | −1.5 |
| 180 | mins | 3.9 | 0.9 | 2.4 | 1.1 | 1.2 | 0.3 | −1.9 |
| 240 | mins | 5.4 | 2.1 | 3.0 | 1.7 | 2.2 | 1.9 | −1.9 |
| 300 | mins | 6.0 | 0.3 | 2.7 | 1.4 | 1.5 | 1.5 | −0.7 |
| 360 | mins | 5.4 | 1.2 | 1.8 | 2.0 | 1.2 | 1.2 | −1.1 |

TABLE 4
Relative Weight Increase of PVBenzoate (Mt/Mo)
After Exposure to Each Analyte Over Time

| Unit: % | | Methanol | Ethanol | Hexane | Toluene | THF | MEK | Ethyl Acetate |
|---|---|---|---|---|---|---|---|---|
| 0 | mins | 0.0 | 0.0 | 0.0 | 0.0 | 0.0 | 0.0 | 0.0 |
| 15 | mins | −3.0 | −5.3 | −5.2 | 0.2 | 24.6 | 5.7 | 8.3 |
| 30 | mins | −1.4 | −2.7 | −5.3 | 2.8 | 41.0 | 16.1 | 18.2 |
| 60 | mins | 1.5 | −2.3 | −4.4 | 7.8 | 56.3 | 28.1 | 35.2 |
| 90 | mins | 2.8 | −1.6 | −4.7 | 12.9 | 65.0 | 33.4 | 44.4 |
| 120 | mins | 2.8 | −1.7 | −4.7 | 17.3 | 74.3 | 35.8 | 47.8 |
| 180 | mins | 2.9 | −1.1 | −4.5 | 25.7 | 81.6 | 41.9 | 54.6 |
| 240 | mins | 3.2 | −0.3 | −4.3 | 28.9 | 86.0 | 45.7 | 58.3 |
| 360 | mins | 2.9 | 0.7 | −4.4 | 35.0 | 93.5 | 52.1 | 65.5 |
| 480 | mins | 3.2 | 1.6 | −4.1 | 38.7 | 98.9 | 56.1 | 66.6 |
| 600 | mins | 3.7 | 2.6 | −4.2 | 40.4 | 108.2 | 59.4 | 70.7 |

TABLE 5
Relative Weight Increase of PVPalmitate (Mt/Mo)
After Exposure to Each Analyte Over Time

| Unit: % | | Methanol | Ethanol | Hexane | Toluene | THF | MEK | Ethyl Acetate |
|---|---|---|---|---|---|---|---|---|
| 0 | mins | 0.0 | 0.0 | 0.0 | 0.0 | 0.0 | 0.0 | 0.0 |
| 5 | mins | 0.9 | 0.9 | 14.2 | 8.4 | 22.8 | 7.4 | 19.7 |
| 15 | mins | 5.7 | 3.7 | 34.2 | 21.7 | 49.5 | 25.8 | 41.0 |
| 30 | mins | 10.3 | 6.9 | 40.9 | 31.6 | 66.6 | 36.3 | 56.6 |
| 60 | mins | 15.9 | 12.5 | 49.0 | 42.4 | 88.9 | 48.0 | 76.9 |
| 120 | mins | 22.9 | 19.2 | 59.2 | 52.6 | 110.2 | 60.4 | 97.5 |
| 180 | mins | 27.4 | 25.3 | 64.8 | 59.7 | 121.5 | 67.8 | 111.8 |
| 240 | mins | 30.3 | 28.0 | 68.0 | 64.1 | 129.6 | 72.0 | 119.9 |
| 300 | mins | 31.7 | 30.5 | 70.3 | 68.1 | 136.5 | 75.9 | 125.4 |
| 360 | mins | 33.6 | 32.6 | 70.4 | 70.8 | 141.2 | 78.7 | 129.5 |
| 420 | mins | 34.2 | 34.1 | 69.5 | 72.8 | 145.4 | 81.4 | 134.0 |
| 480 | mins | 35.7 | 36.4 | 70.3 | 75.0 | 148.0 | 82.7 | 136.7 |
| 540 | mins | 37.1 | 38.0 | 70.1 | 77.1 | 149.8 | 84.1 | 136.9 |
| 600 | mins | 37.3 | 39.0 | 70.8 | 78.1 | 152.0 | 85.8 | 139.5 |

TABLE 6
Relative Weight Increase of PVPivalate (Mt/Mo)
After Exposure to Each Analyte Over Time

| Unit: % | | Methanol | Ethanol | Hexane | Toluene | THF | MEK | Ethyl Acetate |
|---|---|---|---|---|---|---|---|---|
| 0 | mins | 0.0 | 0.0 | 0.0 | 0.0 | 0.0 | 0.0 | 0.0 |
| 5 | mins | 0.9 | 0.9 | 14.2 | 8.4 | 22.8 | 7.4 | 19.7 |
| 15 | mins | 5.7 | 3.7 | 34.2 | 21.7 | 49.5 | 25.8 | 41.0 |
| 30 | mins | 10.3 | 6.9 | 40.9 | 31.6 | 66.6 | 36.3 | 56.6 |
| 60 | mins | 15.9 | 12.5 | 49.0 | 42.4 | 88.9 | 48.0 | 76.9 |
| 120 | mins | 22.9 | 19.2 | 59.2 | 52.6 | 110.2 | 60.4 | 97.5 |
| 180 | mins | 27.4 | 25.3 | 64.8 | 59.7 | 121.5 | 67.8 | 111.8 |
| 240 | mins | 30.3 | 28.0 | 68.0 | 64.1 | 129.6 | 72.0 | 119.9 |
| 300 | mins | 31.7 | 30.5 | 70.3 | 68.1 | 136.5 | 75.9 | 125.4 |
| 360 | mins | 33.6 | 32.6 | 70.4 | 70.8 | 141.2 | 78.7 | 129.5 |
| 420 | mins | 34.2 | 34.1 | 69.5 | 72.8 | 145.4 | 81.4 | 134.0 |
| 480 | mins | 35.7 | 36.4 | 70.3 | 76.0 | 148.0 | 82.7 | 136.7 |
| 540 | mins | 37.1 | 38.0 | 70.1 | 77.1 | 149.8 | 84.1 | 136.9 |
| 600 | mins | 37.3 | 39.0 | 70.8 | 78.1 | 152.0 | 85.8 | 139.5 |

TABLE 7
Relative Weight Increase of PVCinnamate After
Exposure to Each Analyte Over Time (Mt/Mo)

| Unit: % | | Methanol | Ethanol | Hexane | Toluene | THF | MEK | Ethyl Acetate |
|---|---|---|---|---|---|---|---|---|
| 0 | mins | 0.0 | 0.0 | 0.0 | 0.0 | 0.0 | 0.0 | 0.0 |
| 5 | mins | 4.1 | 4.1 | 2.9 | 3.5 | 9.1 | 3.6 | 1.4 |
| 15 | mins | 6.5 | 4.2 | 4.1 | 6.3 | 35.1 | 16.6 | 19.6 |
| 30 | mins | 8.4 | 4.5 | 4.1 | 10.4 | 53.2 | 26.2 | 33.0 |
| 60 | mins | 10.9 | 5.4 | 4.7 | 18.4 | 71.8 | 35.2 | 44.5 |
| 120 | mins | 14.7 | 7.6 | 4.4 | 30.1 | 92.6 | 44.5 | 55.7 |
| 180 | mins | 17.1 | 9.3 | 4.7 | 35.5 | 101.6 | 49.2 | 60.9 |
| 240 | mins | 19.1 | 10.5 | 4.8 | 39.0 | 107.7 | 52.4 | 63.9 |
| 300 | mins | 20.8 | 11.4 | 4.8 | 41.2 | 112.1 | 54.6 | 67.2 |
| 360 | mins | 22.5 | 12.1 | 5.0 | 41.9 | 114.8 | 56.0 | 68.4 |
| 420 | mins | 23.5 | 11.9 | 5.0 | 43.3 | 117.2 | 57.5 | 68.9 |
| 480 | mins | 23.6 | 11.9 | 4.5 | 44.3 | 119.3 | 58.4 | 69.2 |
| 540 | mins | 24.1 | 12.1 | 4.4 | 45.3 | 121.6 | 60.1 | 70.3 |
| 600 | mins | 25.0 | 12.3 | 4.7 | 46.6 | 123.8 | 60.8 | 69.3 |

TABLE 8
The diffusion coefficients (cm²/s) of (a) PVA, (b) PVBenzoate,
(c) PVPalmitate, (d) PVPivalate, and (e) PVCinnamate
are calculated from polymer swelling data.

| | PVBenzoate | PVPalmitate | PVPivalate | PVCinnamate |
|---|---|---|---|---|
| Methanol | — | — | 1.529 | 1.711 |
| Ethanol | — | — | 0.589 | 2.206 |
| Hexane | — | 4.377 | 7.874 | — |
| Toluene | 0.724 | 3.124 | 2.544 | 0.738 |
| THF | 1.931 | 6.664 | 4.589 | 3.201 |
| MEK | 0.976 | 3.190 | 3.791 | 3.447 |
| Ethyl | 0.757 | 4.570 | 2.885 | 3.496 |
| Acetate | | | | |

### Example: Vapor Sensing Tests

### Experimental Setup

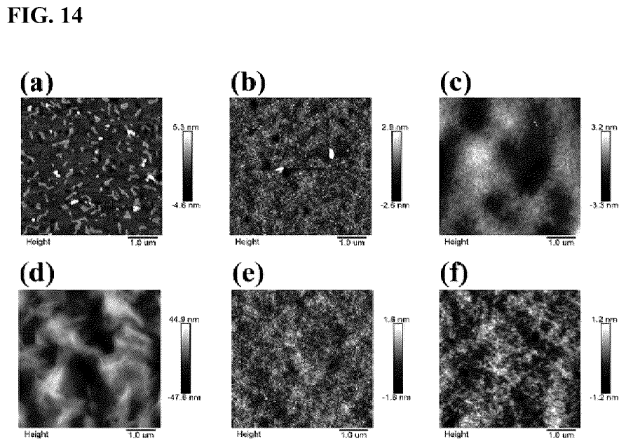

To quantify the effect of VOCs on the electrical response of the CP FET, we designed a bubbler and mass flow controller system to deliver VOCs with known partial pressures in a classified N₂ flow, which was then connected to a probe station chamber for characterization of FET characteristics and sensor current versus time data (FIG. 4). The CP FET fabrication method and AFM analysis results are included in the Experimental section and supporting information (FIGS. 14 and Table 9).

TABLE 9
Roughness of Polymer Film

| | PBTTT | PVA | PVBenzoate | PVPalmitate | PVPivalate | PVCinnamate |
|---|---|---|---|---|---|---|
| RMS | 1.17 nm | 0.85 nm | 0.934 nm | 13.6 nm | 0.472 nm | 0.356 nm |

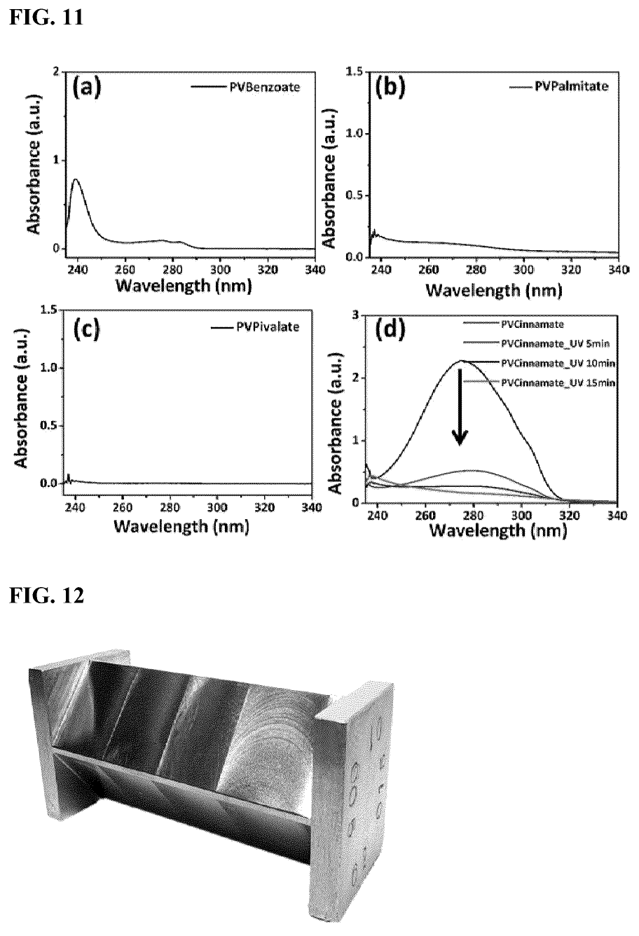

For the membrane deposition, the same film treatment conditions (concentration, spin speed, and heat treatment temperature) were used for each PVA derivative, with the only variable being the solvent. All membrane layers were deposited from butyl acetate solutions, except for PVCinnamate, which was deposited using PGMEA because it is not soluble in butyl acetate. Despite using identical film processing conditions, differences in the inherent rheological properties of each PVA derivative resulted in variations in film thickness, which are summarized in FIG. 11. Film thickness is expected to affect the time it takes for the VOC to pass through the membrane layer and reach the detecting layer, which is a parameter that may affect the electrical response of the sensor. Details regarding film properties will be discussed later in the following section, but some points regarding the effect of film thickness on device properties will now be discussed. The time constants for the response times of each membrane were normalized, and the effect of film thickness on the electrical response was secondary compared to the effect of polymer structure. For example, PVPalmitate film was at least 80% thicker than the other membrane, but on average showed faster response times than the other membrane.

The procedure for the vapor sensing tests is as follows: Before starting each vapor sensor test, the test chamber was purged with dry N₂ for 5 minutes to measure the baseline electrical characteristics. For each device, transfer and output curves were first measured before exposure to analyte to confirm the normal operation of the CP FET vapor sensor and to assess the effect of each membrane deposition on the PBTTT FET characteristics. Then, to analyze the effect of each membrane on the signal response, I_{DS} values were measured at 10-second intervals to characterize the capability of the CP FET sensor to sense vapor over time. The 10-second interval was selected to generate manageable data points for each set of tests, and in the tests that typically last about 40 min, the 10-second interval provided clearly distinguishable differences in the data for different membranes and various analytes. I_{DS} versus time was measured during the vapor sensing test, where V_{DS} and V_{GS} remained constant at −7 V and −4 V, respectively. The reason for using these values is as follows: After the first break-in cycle, a significantly high I_{DS} was measured with negligible changes to I_{D} during repeated test cycles. Higher V_{DS} and V_{GS} increased the initial I_{DS} value and provided a better signal-to-noise ratio in the first cycle, but they also resulted in a decrease in I_{DS} over multiple test cycles as well as damage to the sensor device. Therefore, moderate V_{DS} and V_{GS} values such as −7 V and −4 V were used consistently for all data reported in this study. To initiate each test, I_{DS} signal was allowed to stabilize stabilization during the flow of dry N₂ (10 L/min) for the first 2 min. Then, the flow rate of dry N₂ was reduced to 5 L/min, and the analyte flow line was opened at a rate of 5 L/min, allowing the analyte to evaporate so that the overall flow rate was consistently maintained at 10 L/min during the sensing test. The CP FET vapor sensor was then exposed to various diluted analyte flows for 5 minutes, during which I_{DS} was measured. After the first sensing test, the analyte flow line was closed and the test chamber was purged with dry N₂ at a flow rate of 10 L/min. Two additional analyte/dry N₂ flow cycles were repeated for each analyte, and a total of three cycles were collected for each analyte/membrane combination. Finally, after each set of vapor sensing tests, transfer and output curves were measured again to determine whether analyte exposure degraded the device characteristics. Discussions on the performance and degradation of CP FET are discussed in the last paragraph of the experimental method section.

### Control Experiment

First, a control experiment was conducted using a PBTTT-FET sensor without a membrane to quantitatively analyze the effect of the membrane on the vapor sensing characteristics of the CP FET. The control experiments provide important information on which VOC affects the response of PBTTT-based sensor, which is related to the polarity or solubility between VOC and PBTTT. There are studies by the Arias group that report the Hansen solubility parameters of VOCs and the solubility experiments of PBTTT in various VOCs [37].

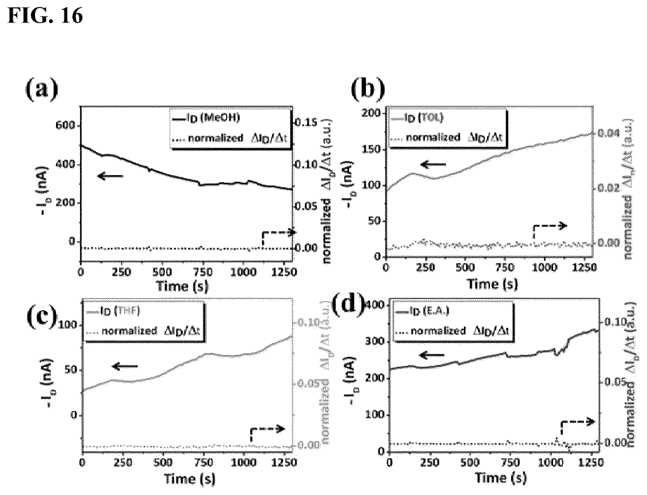

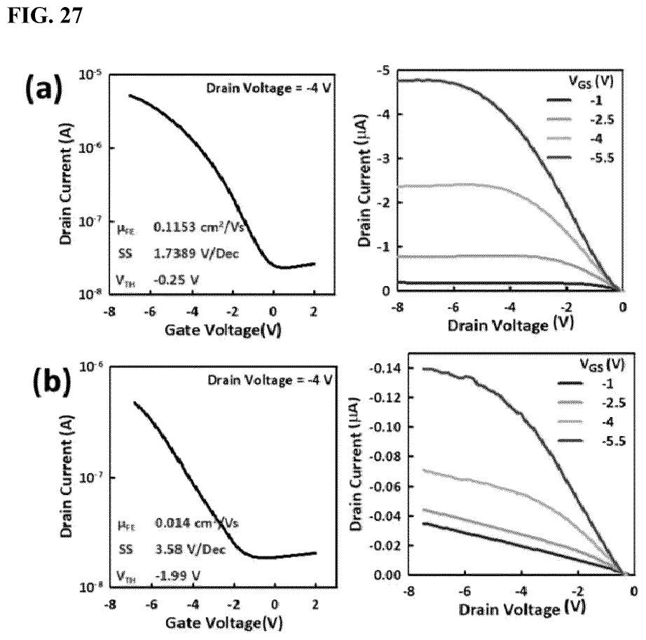

Characterization of the PBTTT CP FET without membrane layers was performed as a control experiment before the vapor sensing test with membranes. In this experiment, normal FET electronic characteristics were measured, in which the hole mobility was 0.1153 cm²/Vs, the subthreshold swing slope was 1.7389 V/Decade, the threshold voltage was 0.25 V, and the current on/off ratio (I_{On/Off} ratio) was 1.81×10². These results are shown in FIG. 27. It should be noted that I_{DS} always decreases when exposed to analytes, even when the analytes are not solvents for PBTTT. For example, methanol and ethanol do not dissolve or swell PBTTT, and they are not expected to significantly permeate the film or alter the π-π stacking shape. Nevertheless, I_{DS} decreases sharply when exposed to these analytes. This indicates that I_{DS} is influenced not only by the swelling phenomenon of the active polymer but also by surface interactions. Another important characteristic of the control data is that the electrical response of the device without a membrane is nearly identical regardless of the type of analyte. The PBTTT detecting layer itself exhibits a general decrease in current regardless of the type of analyte. For comparison with these control devices without membrane layers, an additional control experiment was conducted by depositing known PVA onto the PBTTT vapor sensor without a membrane to determine whether the PVA could reduce VOC transport and thus modulate the response of the PBTTT. As discussed in the introduction, PVA is expected to block vapor from reaching the PBTTT, resulting in a sensor that does not produce a signal upon exposure to VOCs. To verify this phenomenon, only methanol, toluene, THF, and EA were used in the PVA-CP FET vapor sensing tests, and the results are shown in FIG. 16. Compared to the control experiment, I_{DS} shows no meaningful signal change upon detection of these molecules. In particular, PVA was able to block polar VOCs that are similar compounds as PBTTT. These results demonstrate that PVA is suitable for blocking organic vapors because it acts as a low-permeability barrier due to the intermolecular hydrogen bonding forces between PVA chains.

### Data Normalization

From the results of the control experiment, the basic electrical response of the CP FET was observed. For example, concurrently with analyte flow, I_{DS} immediately decreased and then gradually stabilized to a constant value over the next 5 minutes. When the analyte flow was stopped and N₂ was flowed, the I_{DS} increased and tended to reach a saturation point in each cycle, but it generally did not recover to the I_{DS} value observed before the first analyte exposure. However, in the subsequent analyte/flushing cycles, there was little change in the saturated I_{DS} value. In other words, the device exhibited a “start-up effect” during the first cycle, but showed consistent performance thereafter. In addition, it was also confirmed that PVA is an effective barrier against VOCs. Therefore, the PVA derivatives are expected to enhance the selectivity of CP FET vapor sensors by allowing only specific molecules that interact with their functional groups to pass through and reach the PBTTT layer. By comparing experiments with membrane layers, that is, by comparing experiments using various membranes and analytes with no control experiments, it was possible to quantify the effect of the membrane on the vapor sensing response of the sensor.

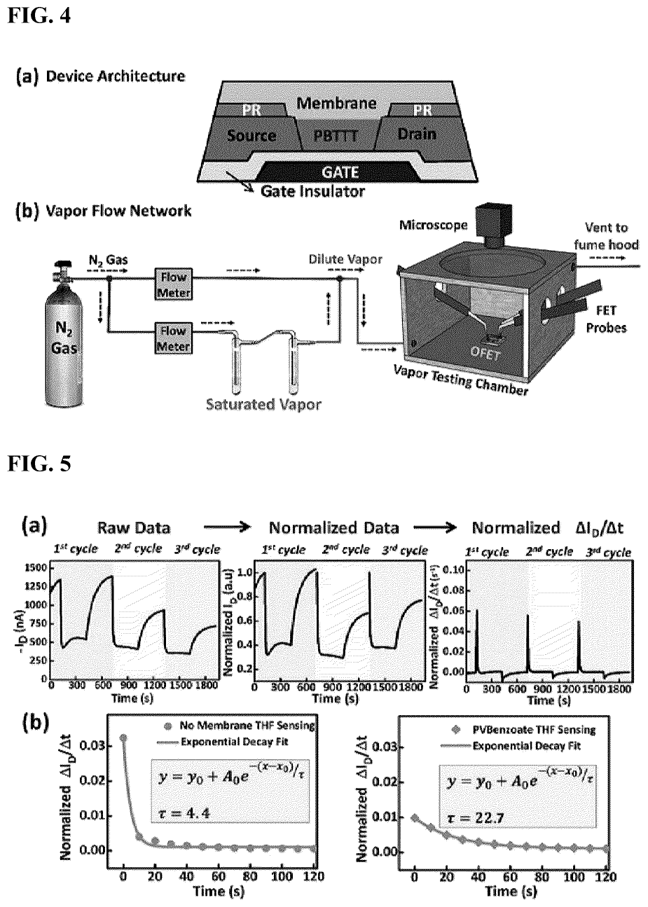

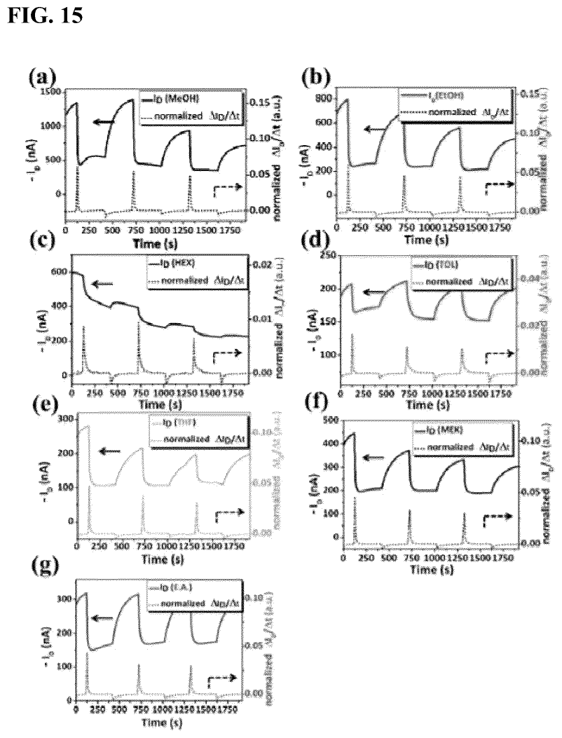

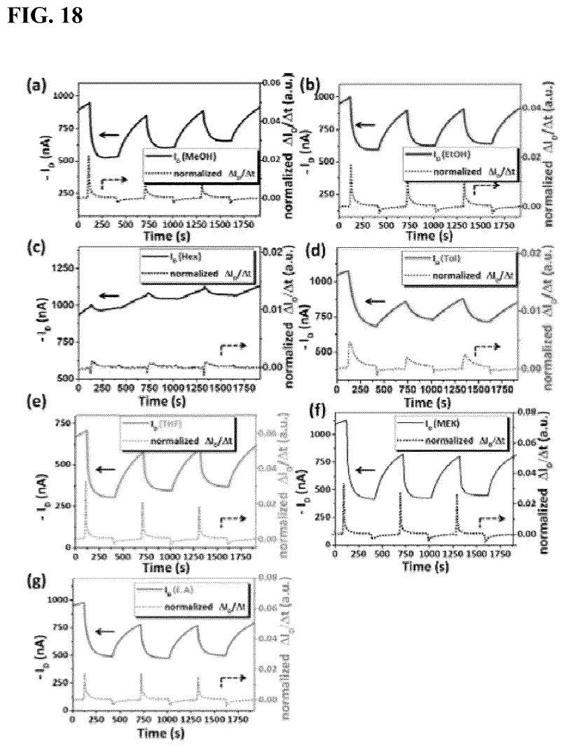

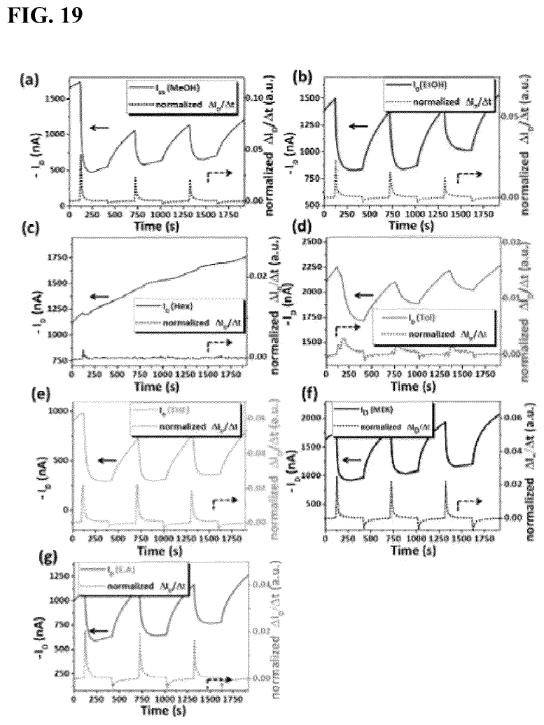

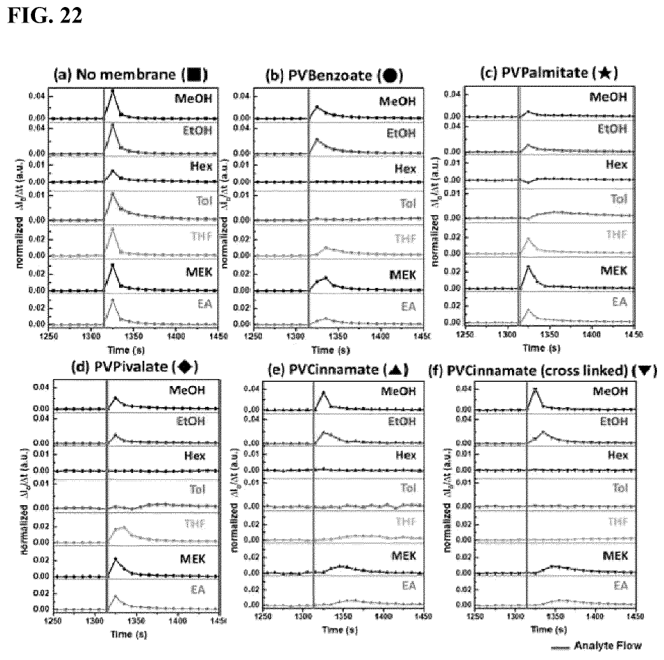

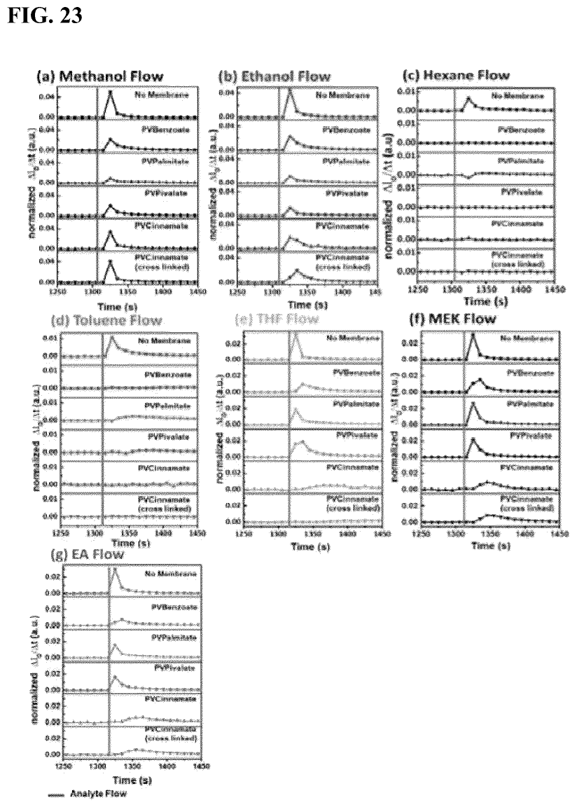

By comparing the raw (unprocessed) data from all the experiments of FIGS. 15 to 21, it was possible to understand the changes in the response of the CP FET vapor sensor to the analytes. However, when comparing only the absolute I_{DS} versus time, it was difficult to quantitatively measure the effect of the membrane because a gradual drift (signal change) in the absolute value of I_{DS} over time and the “start-up effect”, where the current decrease in the first cycle was greater than in subsequent cycles, were observed. To isolate the signal response due to vapor sensing from the drift and start-up effect, the raw data were first divided by the initial current at the beginning of each cycle, and then the time derivative (ΔI_{DS}/Δt) was calculated to compensate for drift. The data was normalized to the saturated current (Iₙₒᵣₘ) at the beginning of each cycle. After normalization, the ΔI_{DS}/Δt value represents the rate of change of current with respect to time, in units of s⁻¹ (FIG. 5(a)). Three cycles were measured consecutively under the same conditions, and each cycle was considered an independent test. For clarity, only the third cycle for ΔI_{DS}/Δt for each analyte/sensor combination was considered in the subsequent drawings (Table 3). Each experiment represented the maximum rate of change or peak value of ΔI_{DS}/t after analyte exposure as mₚₑₐₖ. As shown in FIGS. 22 and 23 and Table 10, mₚₑₐₖ and time (tₚₑₐₖ) taken to reach the maximum value of ΔI_{DS}/t can be easily compared. The exponential decay time constant (τ_{decay}) of ΔI_{DS}/Δt was calculated by fitting an exponential decay function to 14 data points following the initial spike in the third cycle for each analyte/sensor combination (FIG. 5(b), Table 4). This allowed for a quantitative analysis of the signal response resulting from the presence of the membrane. Reproducibility experiments performed without PBTTT using a control experiment. The standard deviations (SD) and standard errors (SE) for mₚₑₐₖ, tₚₑₐₖ, and τ_{decay} were calculated. The SD of m_{peak,methanol} was 0.0118, the SD of m_{peak,ethanol} was 0.0120, the SD of m_{peak,hexane} was 0.0016, the SD of m_{peak,toluene} was 0.0028, the SD of m_{peak,THE} was 0.0064, the SD of m_{peak,MEK} was 0.0129, and the SD of m_{peak,EA} was 0.0080. The SD of t_{peak,methanol}, t_{peak,ethanol}, t_{peak,toluene}, t_{peak,THF}, t_{peak,MEK}, t_{peak,EA} was 0.0 s, while t_{peak,hexane} was 5.5 s. The SD of τ_{decay,methanol} was 1.4 s, the SD of τ_{decay,ethanol} was 0.8 s, the SD of τ_{decay,hexane} was 6.9 s, the SD of τ_{decay,toluene} was 4.3 s, the SD of τ_{decay,THF} was 1.2 s, the SD of τ_{decay,MEK} was 1.0 s, and the SD of τ_{decay,EA} was 1.3 s. By comparing the standard errors of these measurements with those from the subsequent experiments, it was found that mₚₑₐₖ, tₚₑₐₖ, and τ_{decay} all showed large changes several times greater than the SE for each analyte, and this allows for the calculation of confidence intervals and confirms that the membrane induced statistically significant changes in the electrical response of the sensor.

TABLE 10
Time Required to Reach mₚₑₐₖ(tₚₑₐₖ) After Analyte
Flow For Each Analyte-Membrane Combination

| | No | | | | | PVCinnamate |
|---|---|---|---|---|---|---|
| tₚₑₐₖ (s) | Membrane | PVBenzoate | PVPalmitate | PVPivalate | PVCinnamate | (cross-linked) |
| Methanol | 10 | 10 | 10 | 10 | 10 | 10 |
| Ethanol | 10 | 10 | 10 | 10 | 10 | 20 |
| Hexane | 10 | — | 10 | — | — | — |
| Toluene | 10 | — | 40 | — | — | — |
| THF | 10 | 20 | 10 | 20 | 50 | — |
| MEK | 10 | 20 | 10 | 10 | 30 | 30 |
| Ethyl Acetate | 10 | 20 | 10 | 10 | 50 | 40 |

### Example 5: Effect of Membrane on CP FET Vapor Sensor Exposed to Various Analytes

Next, it is determined whether PVA-based membranes functionalized with various molecular groups could enable the PBTTT vapor sensor to respond selectively to different types of analytes. Five different PVA derivatives with unique functional groups were characterized. The effect of each membrane may be related to the unique functional group of each membrane. For example, by comparing the properties of PVBenzoate (with a benzene ring in its side chain) and PVPalmitate (with a C15 aliphatic group in its side chain), the effects of aromatic rings and aliphatic groups may be evaluated. By comparing PVPalmitate and PVPivalate, the differences between long linear and short-branched aliphatic chains may be compared. In addition, the data from PVCinnamate allow for the evaluation of the specific effects of the π-conjugated system. Finally, the effect of cross-linking in the membrane may be assessed through the results of photopolymerized PVCinnamate.

### Methanol and Ethanol Sensing

FIG. 23 shows −ΔI_{DS}/Δt versus time for the third test cycle of the CP FET sensor. FIG. 23(a) shows the methanol sensing data for all CP FET sensors, and FIG. 23(b) shows the ethanol sensing data for all CP FET sensors. Table 4 shows that, for sensors including all membranes, the time required for methanol and ethanol to reach saturation in the detecting layer increases, resulting in a higher increase in τ_{decay}. For example, in the case of methanol, τ_{decay} increased from 5.2 s without PVPalmitate to 11.2 s with PVPalmitate, and for ethanol-sensing PVPalmitate, τ_{decay} increased from 6.4 s to 14.4 s. The standard errors of τ_{decay,methanol}, and τ_{decay,ethanol} for PVPalmitate were 0.6 s and 1.5 s, respectively, indicating that the difference in τ_{decay} for these two analytes was statistically significant. Another interesting finding was that only the cross-linked PVCinnamate-CP FET sensor exhibited a delay in tₚₑₐₖ for ethanol, which occurred 20 s after the device was exposed to the ethanol flow, compared to 10 s for all other devices. It can be interpreted that ethanol diffuses more slowly through the cross-linked PVCinnamate membrane compared to other membranes. Comparison of methanol and ethanol sensing data suggests that an appropriate membrane can enhance sensing selectivity, even for compounds with similar compound properties and structures.

### Hexane Sensing

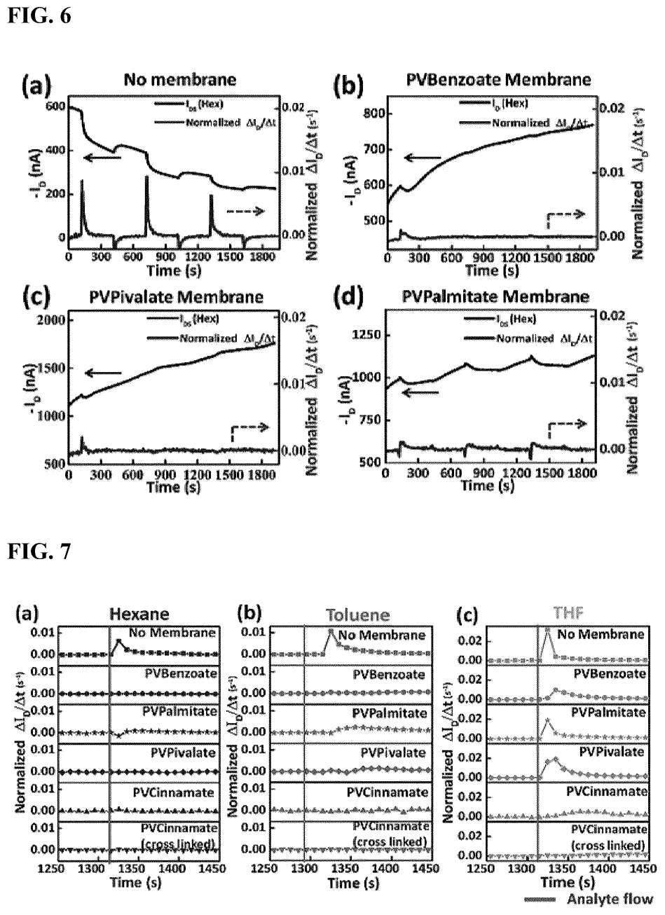

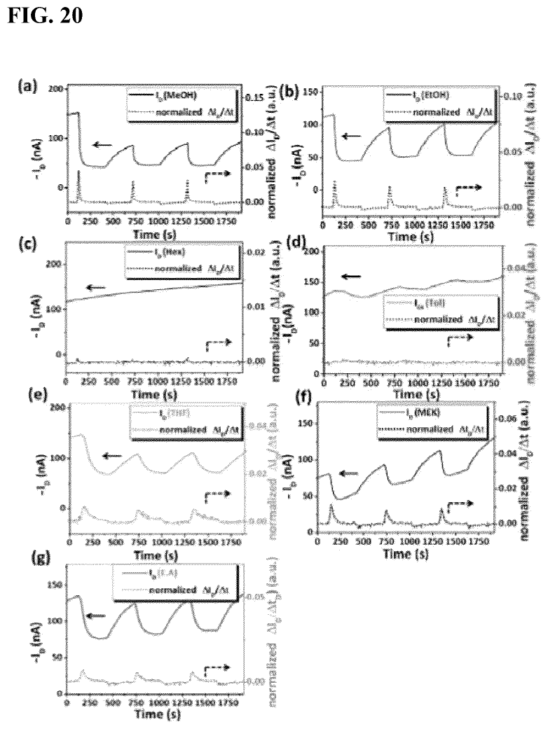

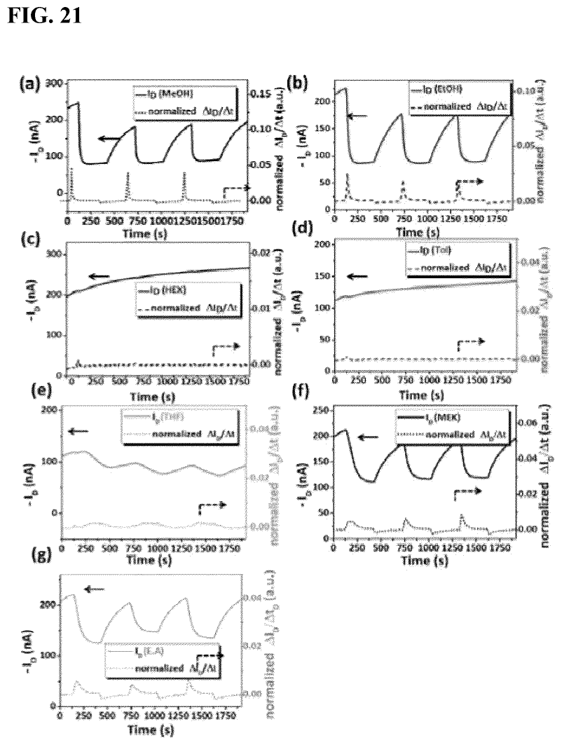

FIG. 6 shows raw data and time versus −ΔI_{DS}/Δt data for all cycles upon exposure to hexane vapor. This includes the case of devices including membrane-free CP FET, PVBenzoate-CP FET, PVPivalate-CP FET, and PVPalmitate-CP FET. FIG. 6(a) shows that CP FET made with PBTTT can detect hexane. Hexane has a low polarity and poor solubility in PBTTT. FIGS. 6(b) and 6C show data for PVBenzoate and PVPivalate membrane sensors. A strong blocking effect against hexane is observed with these membranes. Both the PVCinnamate and cross-linked PVCinnamate sensors also exhibited excellent blocking effects during hexane sensing, as shown in FIGS. 20(c) and 21(c). Data for the PVPalmitate-CP FET exposed to hexane are shown in FIG. 6(d). Interestingly, PVPalmitate shows a weak response compared to the data of the CP FET without a membrane, but it still indicates that hexane can pass through. This can be reasonably explained by considering the similar compound structures of PVPalmitate's long, linear aliphatic chains and hexane. This is because it diffuses more easily than in the other membranes (FIG. 7(a)). Another interesting aspect of PVPalmitate-CP FET is the steadily increasing I_{DS} observed 10 seconds after hexane flow, which occurs only with this membrane and results in a negative value of m_{peak,hexane} in Table 3. The standard deviation and standard error (SE) of these measurement were small (m_{peak,hexane} was 0.0002 and 0.0001, respectively). This shows that the data are reproducible and that the presence of the membrane causes a significant change in these values.

### Toluene Sensing

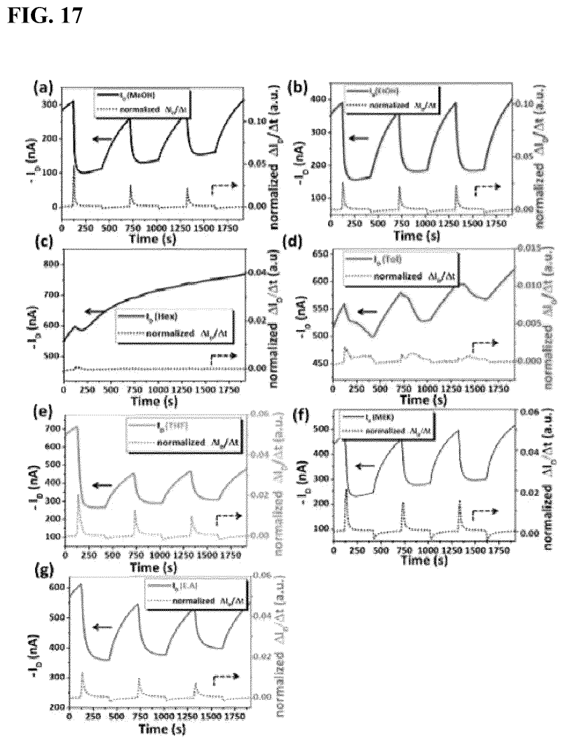

Toluene-sensing data for CP FET without a membrane showed a similar signal response and typical −ΔI_{DS}/Δt values when compared to the other analytes. When a membrane is deposited on the CP FET, the signal is significantly decreased when the sensing test is conducted (FIG. 7(b)). However, the raw data of I_{DS} versus time from CP FET vapor detection data show that, unlike hexane, toluene is not completely blocked. These data also show that the I_{DS} versus t and ΔI_{DS}/Δt versus t plots for each membrane are unique in shape, and the same shape appears consistently across all analyte flow cycles. This means that the presence of a membrane gives a much more unique signal characteristics for the analyte compared to the case without a membrane. In FIG. 7(b), it appears that the PVBenzoate sensor does not exhibit a strong response to toluene, but a closer examination of the detailed data in FIG. 17(d) reveals a clear and gradual triangular wave-shaped response to toluene vapor. For example, compared to the sensing of hexane by PVPalmitate, this gradual sensing of toluene may occur because PVPalmitate includes a long C16 aliphatic chain with only weak van der Waals forces. PVBenzoate includes a small benzoate group with stronger π-π0 and dipole forces, which accounts for the observed behavior. It is expected that various analyte/membrane combinations such as toluene/PVBenzoate may be numerically identified through appropriate signal recognition algorithms based on machine learning or artificial intelligence. However, applying AI to signal processing is still a task for the future, as it is beyond the scope of the present work. In addition, the effect of the membrane can be confirmed by comparing the extracted data. For example, in Table 4, the τ_{decay,toluene} of PVPalmitate is 76.7 s (SD: 9.7 s, SE: 5.6 s), which is much higher than the τ_{decay,toluene} of the sensor without a membrane.

### THF Sensing

THF sensing data show that, compared to hexane and toluene, the membrane does not provide a significant blocking effect against THF, which is an excellent solvent for all polymer membranes. However, as shown in FIG. 7(c), t_{peak,THF} of PVBenzoate and PVPivalate sensors exhibits a 10-second delay from the maximum value after THE flow. In addition, τ_{decay,THF} of PVBenzoate is 25.4 seconds (SD: 4.3 seconds, SE: 2.5 seconds), and the PVPivalate sensor is 13.1 seconds (SD: 1.3 seconds, SE: 0.8 seconds), both of which are much higher than that of the PVPalmitate device. This indicates that while THF rapidly passes through PVBenzoate, PVPivalate, and PVPalmitate, its diffusion through the PVBenzoate and PVPivalate membranes is slower than through the PVPalmitate membrane, resulting in an increased time for THF to saturate the PBTTT layer. m_{peak,THF},t_{peak,THF}, and τ_{decay,THF} demonstrate that the presence of the membrane in the vapor sensor not only reduces the amount of analyte diffusing through, but also affects the time it takes for the analytes to saturate the sensor signal.

### MEK and EA Sensing

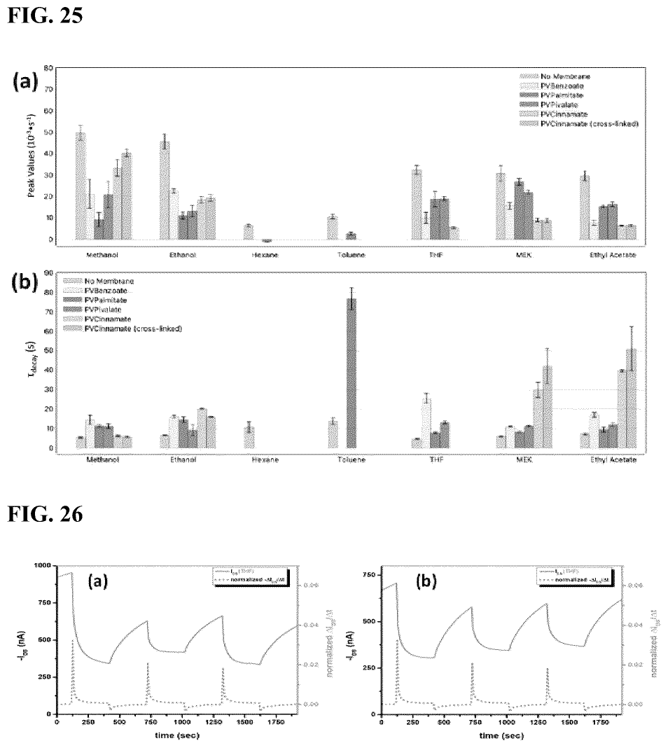

In FIGS. 23(c) and 23(g), PVBenzoate, PVCinnamate, and cross-linked PVCinnamate-based sensors exhibited different responses from the sensor without a membrane during the sensing of MEK and EA. For example, the presence of PVBenzoate increased t_{peak,MEK} and t_{peak,EA} from 10 s to 20 s (SD: 0.0 s, SE: 0.0 s). A good example of quantification-based comparison is τ_{decay}. τ_{decay,EA} of PVCinnamate is 39.5 s (SD: 1.1 s, SE: 0.6 s), which is more than 30 seconds higher than τ_{decay,EA} of the sensor without a membrane. Similarly, the data in Tables 3 and 4 and FIGS. 25 and 26 allow for the numerical confirmation that a distinct signal response is generated due to the presence of the membrane. However, it was difficult for the five types of membranes used in our experiments to distinguish the responses to MEK and EA as clearly as they did for other analyte comparisons. This is because the mₚₑₐₖ, tₚₑₐₖ, and τ_{decay} values do not show significant difference between these two analytes. However, it is expected that it may be possible in the future to distinguish between these two analytes by using different membrane combinations.

TABLE 11
Normalized Exponential Decay Time Constant (τ_{decay}) According to Film Thickness

| Normalized | | | | | PVCinnamate |
|---|---|---|---|---|---|
| τ_{decay} (s/nm) | PVBenzoate | PVPalmitate | PVPivalate | PVCinnamate | (cross-linked) |
| Methanol | 0.091 | 0.016 | 0.029 | 0.019 | 0.017 |
| Ethanol | 0.101 | 0.020 | 0.023 | 0.063 | 0.050 |
| Hexane | — | — | — | — | — |
| Toluene | — | 0.109 | — | — | — |
| THF | 0.160 | 0.011 | 0.034 | — | — |
| MEK | 0.069 | 0.011 | 0.029 | 0.094 | 0.133 |
| Ethyl | 0.107 | 0.013 | 0.031 | 0.125 | 0.161 |
| Acetate | | | | | |

### Comparison of PVCinnamate vs. Cross-Linked PVCinnamate

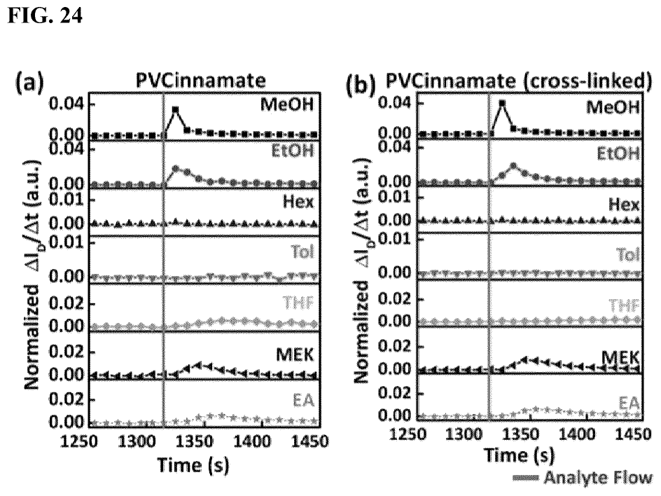

A comparison of ΔI_{DS}/Δt between PVCinnamate-CP FET and cross-linked PVCinnamate-CP FET is shown in FIG. 24, providing information on the effect of cross-linking in the polymer membrane. One of the most important differences is the time it takes for ΔI_{DS}/Δt to reach its maximum value during the detection of ethanol. The PVCinnamate-based sensor took 10 seconds to reach the maximum value, whereas the cross-linked film required 20 seconds. This shows that cross-linking can significantly affect the signal response and enhance the selectivity of the sensor. In this case, methanol and ethanol can be clearly distinguished by using the cross-linked PVCinnamate membrane. By comparing the initial slopes for methanol and ethanol (0.0403 s⁻¹ vs. 0.0192 s⁻¹; 6σ) and the decay constants (5.5 s vs. 15.9 s; 26σ), it can be confirmed that the cross-linked PVCinnamate membrane in particular can distinguish between ethanol and methanol with high reliability. Examining the raw I_{DS} vs t data of FIGS. 20(d), 20(e), 21(e), and 21(e), it can be confirmed that the cross-linked membrane more effectively blocked or delayed the response to toluene and THF. A diagram summarizing responses to various analytes in a sensor array using various membranes is included in FIG. 25. In particular, each analyte generates a unique response pattern in the sensor array with different membranes, and at least one membrane shows a difference of more than 2σ compared to the other analytes, allowing each analyte to be identified with high reliability.

### Performance and Reliability of CP FET

All PBTTT devices exhibited typical FET behavior (transfer and output curves) both before and after exposure to analyte vapors. The hole mobility of the CP FET used without a membrane was 0.1153 cm²/Vs, and the subthreshold voltage was −0.25 V. After testing for seven analytes for 6 h, the hole mobility decreased to 0.0141 cm²/Vs and the subthreshold voltage was changed to −1.99 V, but the decrease was similar to that observed when the FET device was tested repeatedly in the absence of VOCs. These changes represent the decrease that occurred for approximately 6 h or more after exposure to various analytes and electrical stresses. However, the transfer and output curves shown in FIGS. 26 and 27 indicate that the device still exhibits normal output and transfer curves, and the normalized data still show consistent mₚₑₐₖ and τ_{decay} values.

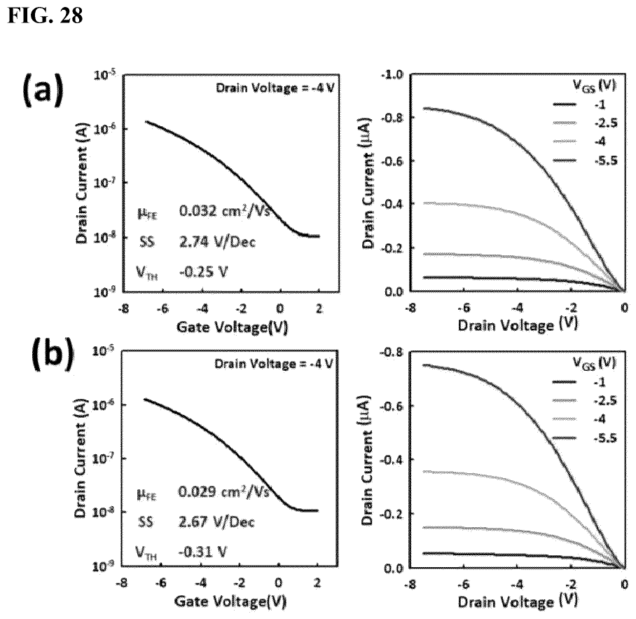

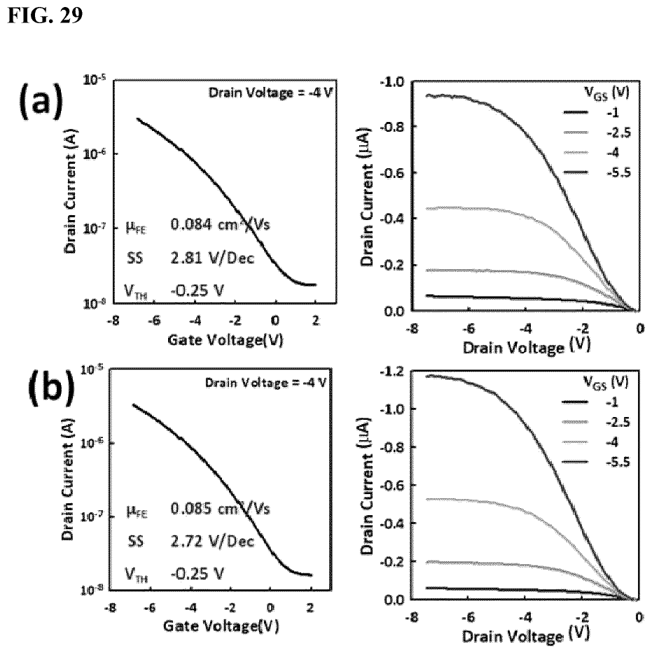

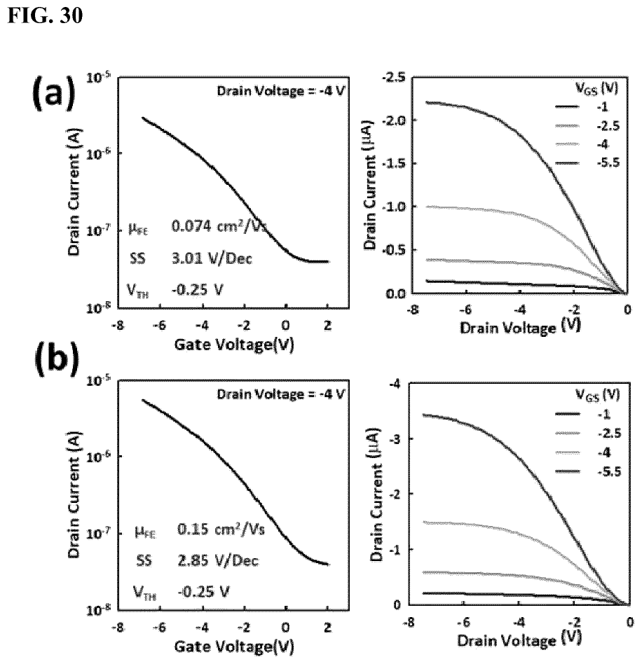

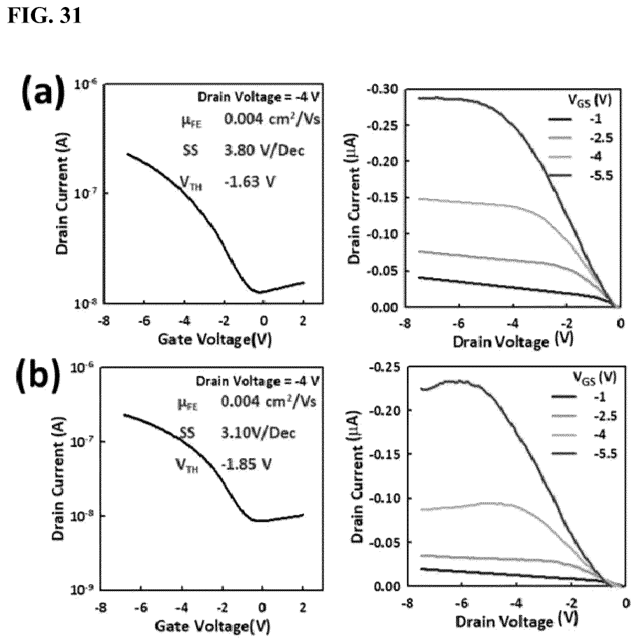

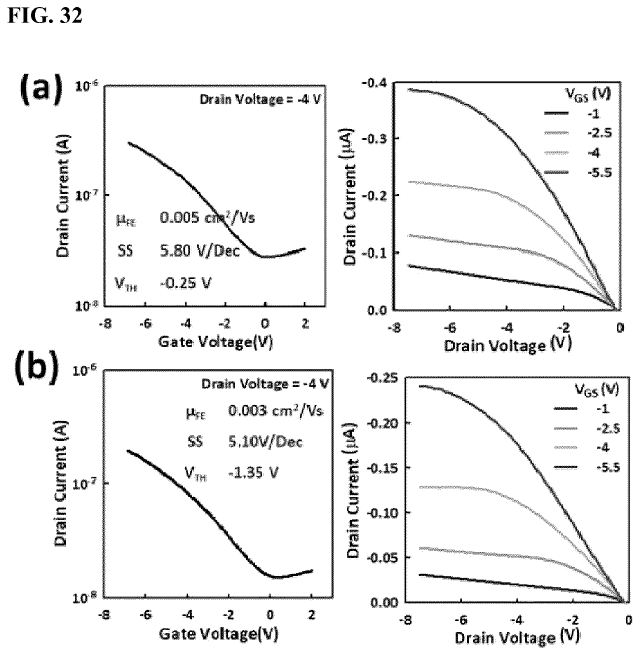

In FIGS. 28 to 32, the CP FET exhibits normal FET characteristics both before and after membrane deposition. All devices with membranes exhibited typical FET characteristics both before and after membrane deposition, as well as before and after vapor sensing tests. In particular, all FETs with membranes exhibited slightly lower mobility compared to devices without a membrane, but the decrease in mobility and FET characteristics was smaller than in FETs without a membrane (FIGS. 27 to 32).

In one aspect, in order to enhance the selectivity of the PBTTT-OFET vapor sensor, a fast, simple, and economical synthesis method for manufacturing various PVA derivatives has been developed. The properties of these polymers were characterized, and by using them as membrane layers in PBTTT-OFET vapor sensors, how membranes with various functional groups influence the signal responses and whether employing such types of membranes can enhance the selectivity of gas sensors were investigated. It was confirmed that, when unmodified PVA was applied to the PBTTT FET, it effectively suppressed the response of the PBTTT sensor to VOC. This means that a highly impermeable barrier to VOC is formed due to the highly polar structure of PVA and the strong hydrogen bonding between PVA chains. The present disclosure suggests the possibility of modulating the response of a vapor sensor using a modified PVA membrane with higher permeability than PVA and selective permeability to specific analytes.

CP FET sensors used without a membrane showed little difference in signal responses for almost all analytes, whereas CP FET with PVA derivative membranes generated a unique set of responses for each analyte. Certain membranes were able to distinguish specific analytes; for example, PVPalmitate generated a unique signal upon exposure to hexane while others did not; and cross-linked PVCinnamate was able to distinguish methanol and ethanol with 100% confidence. Therefore, the sensor array with PVA derivatives described in this study, which used PBTTT only, was able to uniquely identify one of the seven analytes used in this study based on a unique set of electrical responses to randomly selected molecules among various analytes. Membrane-free PBTTT sensors showed the same general response to various analytes.

Each membrane had a unique response to a variety of analytes, but the details of the specific interactions between analytes and polymers require further research on binding energies derived from van der Waals forces, diatoms, hydrogen bonds, molecular geometry, and the like. Computational techniques such as density functional theory have demonstrated the ability to model and quantify these interactions, and can help to tune the molecular structure of polymer membranes to detect and identify specific analytes. Therefore, it may help to regulate these interactions to optimize the performance of membrane-based gas sensors.

The use of a wider variety of membranes and various polymers other than PBTTT is expected to make it easier to fabricate large-area arrays capable of distinguishing analytes beyond the seven analytes used herein, thereby enabling the realization of a functional electronic nose with capabilities similar to many olfactory cells found in biological systems. It is expected that this strategy, when combined with appropriate signal processing and machine learning software, could pave the way for realizing functional electronic noses.

While specific portions of the present disclosure have been described in detail above, it will be apparent to those skilled in the art that such specific descriptions are merely preferred embodiments and that the scope of the present disclosure is not limited thereto. Therefore, it will be said that the practical scope of the present disclosure is defined by the claims and their equivalents.

1. An acylated polyvinyl alcohol compound represented by Chemical Formula 1: 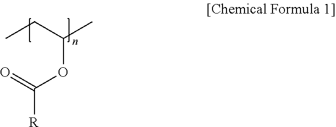
where, R is 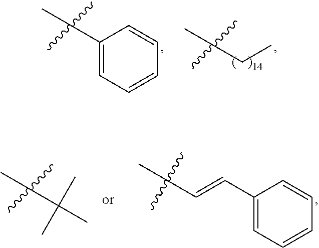
and n is an integer from 50 to 5000.

2. A cross-linked acylated polyvinyl alcohol compound represented by Chemical Formula (2): 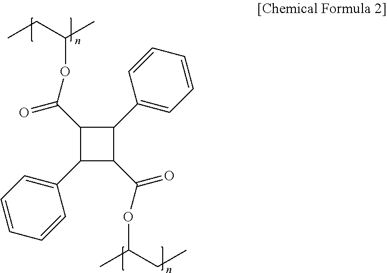
where, n is an integer from 50 to 5000.

3. A method for preparing an acylated polyvinyl alcohol compound of Chemical Formula 1, the method including the steps of:
(a) dissolving a polyvinyl alcohol of Chemical Formula 3 in a solvent in a microwave reactor; and
(b) adding a base and an acyl chloride of Chemical Formula 4 to the dissolved polyvinyl alcohol and allowing them to react; 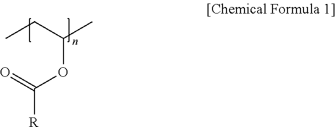
where, R is 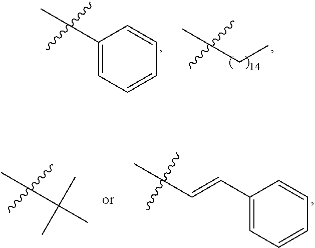
and n is an integer from 50 to 5000, 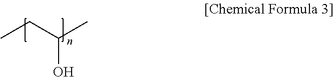
where, n is an integer from 50 to 5000, and

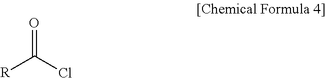

4. The method according to claim 3, wherein the solvent of the step (a) is one or more selected from a group consisting of 1-methylimidazole, N-methylpyrrolidone, dimethyl formamide, and dimethyl sulfoxide.

5. The method according to claim 3, wherein a reaction time of the step (b) is 5 minutes to 24 hours.

6. A method for preparing a cross-linked acylated polyvinyl alcohol compound of Chemical Formula 2, comprising a step of cross-linking a poly (vinyl cinamate) of Chemical Formula 1-4 by irradiating it with UV light: 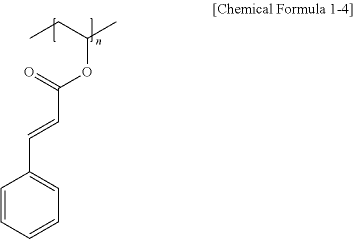
where, n is an integer from 50 to 5000; 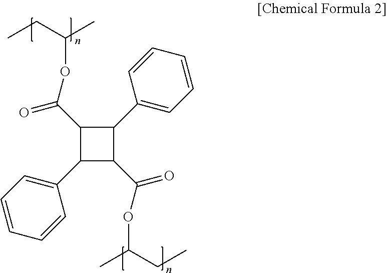
where, n is an integer from 50 to 5000.

7. A chemical sensor, comprising:
an organic field-effect transistor (OFET); and
a polymer membrane coated with the compound of claim 1 on the OFET.

8. The chemical sensor according to claim 7, wherein a thickness of the polymer membrane is 50 nm to 1 μm.

9. A method for preparing a chemical sensor, comprising a step of coating an OFET with the compound of claim 1.

10. A method for preparing a chemical sensor, comprising a step of coating an OFET with the compound of Chemical Formula 1-4 and then irradiating the OFET with ultraviolet light.

11. The method according to claim 9, further comprising a step of performing heat treatment at a temperature of 80 to 150° C. for 15 to 30 minutes after the coating.

12. A method for detecting a chemical species using a chemical sensor, the method comprising a step of bringing the chemical sensor according to claim 7 into contact with the chemical species and detecting the chemical species by measuring a drain current over time.

13. The method according to claim 12, wherein the chemical species includes volatile organic compounds (VOCs).

14. A chemical sensor, comprising:
an organic field-effect transistor (OFET); and
a polymer membrane coated with the compound of claim 2 on the OFET.

15. The method according to claim 10, further comprising a step of performing heat treatment at a temperature of 80 to 150° C. for 15 to 30 minutes after the coating.

16. The chemical sensor according to claim 14, wherein a thickness of the polymer membrane is 50 nm to 1 μm.

17. A method for detecting a chemical species using a chemical sensor, the method comprising a step of bringing the chemical sensor according to claim 14 into contact with the chemical species and detecting the chemical species by measuring a drain current over time.

18. The method according to claim 17, wherein the chemical species includes volatile organic compounds (VOCs).
